SEQUENCE LISTING (8 sequences)

SEQ ID NO: 1 - DNA, 80 bp, synthetic construct
  source 1..80: mol_type = other DNA
tcgtcggcagcgtcagatgtgtataagagacagaagtctgtactatggnnnnnnnntttttttttttttttttttttttt

SEQ ID NO: 2 - DNA, 57 bp, synthetic construct
  source 1..57: mol_type = other DNA
tcgtcggcagcgtcagatgtgtataagagacagtttttttttttttttttttttttt

SEQ ID NO: 3 - DNA, 33 bp, synthetic construct
  source 1..33: mol_type = other DNA
tcgtcggcagcgtcagatgtgtataagagacag

SEQ ID NO: 4 - DNA, 13 bp, synthetic construct
  source 1..13: mol_type = other DNA
aaaaaaaaaaaaa

SEQ ID NO: 5 - DNA, 11 bp, synthetic construct
  source 1..11: mol_type = other DNA
attgcgcaatg

SEQ ID NO: 6 - DNA, 19 bp, synthetic construct
  source 1..19: mol_type = other DNA
ctgtctcttatacacatct

SEQ ID NO: 7 - protein, 60 aa, Homo sapiens
LNDQFHAIFAKERGELTEKNRMGDKLCDFVRSVSSRLAQIASIAQSFAELNVSVNIVDAM

SEQ ID NO: 8 - DNA, 100 bp, Homo sapiens
  source 1..100: mol_type = other DNA
tcacgtaggttcagtgggcggaagaggtggccccggatgctgcggcgcccgctggccgggctggctgcggccgccctgggccgggccccaccggacggtg

(19) United States
(12) Patent Application Publication
(10) Pub. No.: US 2024/0344109 A1
(43) Pub. Date: Oct. 17, 2024

(54) METHODS OF DETERMINING THE NUMBER OF COPIES OR SEQUENCE OF ONE OR MORE RNA MOLECULES

(71) Applicant: BASIC GENOMICS AB, Solna (SE)

(72) Inventors: Gerardus Johannes Hendriks, Solna (SE); John Anton Magnus Larsson, Solna (SE); Thore Rickard Håkan Sandberg, Solna (SE)

(21) Appl. No.: 18/294,215

(22) PCT Filed: Jul. 29, 2022
(86) PCT No.: PCT/EP2022/071372

§ 371 Date: Feb. 1, 2024

(30) Foreign Application Priority Data
Aug. 3, 2021 (GB) 2111184.4

(51) Int. Cl. C12Q 1/6806

(52) U.S. Cl. CPC C12Q 1/6806

(57) ABSTRACT

The present invention relates to a method of determining the number of copies of one or more RNA molecules in a population of RNA molecules and a method of determining the sequence of one or more RNA molecules in a population of RNA molecules, wherein the methods include a step of converting the population of RNA molecules to a population of DNA molecules comprising one or more base conversion, by error-prone reverse transcription. The present invention also relates to a population of DNA molecules obtained or obtainable by the methods disclosed herein.

The present invention relates to a method of determining the number of copies of one or more RNA molecules in a population of RNA molecules, and to a method of determining the sequence of one or more RNA molecules in a population of RNA molecules, wherein the methods include a step of converting the population of RNA molecules to a population of DNA molecules by error-prone reverse transcription. The present invention also relates to a population of DNA molecules obtained or obtainable by the methods disclosed herein.

Massively parallel sequencing applications have transformed biology and medicine. To investigate genetic programs in cell populations or even single cells, it is today routine to carry out RNA-sequencing on thousands to millions of individual single cells or on cell populations. Such analysis can reveal patterns of gene, isoform and allelic expression across cell types and states. However, current short-read single-cell RNA-sequencing (scRNA-seq) methods have limited ability to count RNAs at allele and isoform resolution, and long-read sequencing techniques are prohibitively expensive for the depth required for large-scale applications across cells.

Most scRNA-seq methods count RNAs by sequencing a unique molecular identifier (UMI) together with a short part of the RNA (from either the 5′ or 3′ end). Those RNA end counting strategies have been effective in estimating gene expression across large numbers of cells, while controlling for PCR amplification biases, yet RNA end sequencing provides limited coverage of transcribed genetic variation and transcript isoform expression.

Most contemporary sequencing protocols are built upon short-read sequencing platforms (e.g. those of Illumina or MGI) as these mature platforms are cost-effective for deep large-scale sequencing across cells. Transcriptomic analysis using short-read sequencing technologies has a common limitation in that the individual short-reads can only cover a fraction of the body of a particular transcript. The short fragments generated by commonly used droplet methods (e.g. 10× Chromium systems) target either of the ends of an RNA transcript (i.e. the 3′ end or the 5′ end, depending on protocol used). Alternatively, short reads can be distributed all throughout the RNA transcripts as in Smart-seq2 (Picelli et al, 2013. Nature Methods, 10:1096-1098) or Smart-seq3 (Hagemann-Jensen et al, 2020. Nature Biotechnology, 38:708-714) technologies. However, even for methods with reads distributed across RNA transcripts, the large number of short reads (or paired-end read pairs) cannot be individually assembled (e.g. to reconstitute the original sequences of individual molecules). Instead, the total read coverage across transcripts relates to the total number of RNA transcripts sequenced from the cell(s). Importantly, in the methods described above, molecular counting through the use of a Unique Molecular Identifier (UMI) is always either restricted to the 3′ or 5′ end of the complementary DNA (cDNA) molecules. Combining reads covering the same unique molecular barcode can provide limited RNA sequence reconstruction (Hagemann-Jensen et al, 2020. Nature Biotechnology, 38:708-714), theoretically up to the maximum fragment length that can be sequenced on a short-read sequencing instrument (for example, 200-800 base pairs for Illumina sequencing).

Sequencing of full-length RNA transcripts using long-read DNA sequencing technologies (e.g. using Pacific Biosystem reaction reactors or Oxford nanopore sequencing) can directly quantify allele and isoform-level expression, yet their current cost relative to read depths hinder their broad application across cells, tissues, and organisms. Furthermore, such long-read sequencing platforms are more costly and do not offer the same level of parallelisation in terms of the number of DNA molecules that can be sequenced simultaneously in short-read platforms.

Thus, there exists a need for methods of counting and/or qualitatively sequencing RNA molecules that address the shortcomings encountered in prior art methods.

The present inventors have developed a new approach for counting and/or qualitatively sequencing RNA molecules in a population, which addresses the above problems.

As discussed in detail below, the inventors' approach involves introducing unique patterns of base-conversion into individual cDNA molecules during reverse transcription of corresponding RNA molecules in a population, and then using those unique patterns to count individual RNA molecules in a population, and also assemble sequences from short reads. The inventors have surprisingly found that the unique patterns of base-conversion can be stably propagated during subsequent DNA amplification, and can be used to identify and count individual transcripts present in the population of RNA molecules. Due to the unique nature of each base-conversion pattern in a given molecule in the starting plurality of cDNA molecules, the inventors are able to simultaneously sequence and count much larger numbers of transcripts in a population of RNA molecules than is possible using existing short-read sequencing technologies. Advantageously, the method of the present invention also identifies the origin of the analysed sequencing reads as being RNA transcribed from the positive strand, RNA transcribed from the negative strand (together referred to as “strandedness”), or any DNA source (such as, for example, genomic DNA).

In a first aspect, the invention provides a method for determining the number of copies of one or more RNA molecule in a population of RNA molecules, comprising the steps of:
(i) providing a population of RNA molecules;
(ii) subjecting the population of RNA molecules to error-prone reverse transcription, to generate a population of DNA molecules in which each DNA molecule comprises one or more base-conversions relative to the corresponding RNA molecule, and wherein each DNA molecule comprises a molecule-specific base-conversion pattern;
   and using the molecule-specific base-conversion pattern to determine the number of copies of the one or more RNA molecule in a population.

In some embodiments of the first aspect, the step of using the molecule-specific base-conversion pattern to determine the number of copies of the one or more RNA molecule in a population further comprises the following, performed after Step (ii):
(iii) determining the sequence of overlapping fragments of DNA molecules in the population;
(iv) determining, from the information in Step (iii), the partial or full-length sequence of the DNA molecules in the population, by assembling the sequence of overlapping fragments based on the molecule-specific base-conversion pattern in the DNA molecules;
(v) determining, from the information in Step (iv), the sequence of the RNA molecules which correspond to the DNA molecules; and
(vi) determining, from the information in Step (v), the number of copies of one or more RNA molecule in the population.

In a second aspect, the invention provides a method for determining the number of copies of one or more RNA molecule in a population of RNA molecules, comprising the steps of:
(i) providing a population of RNA molecules;
(ii) subjecting the population of RNA molecules to error-prone reverse transcription, to generate a population of DNA molecules in which each DNA molecule comprises one or more base-conversions relative to the corresponding RNA molecule, and wherein each DNA molecule comprises a molecule-specific base-conversion pattern;
(iii) determining the sequence of overlapping fragments of DNA molecules in the population;
(iv) determining, from the information in Step (iii), the partial or full-length sequence of the DNA molecules in the population, by assembling the sequence of overlapping fragments based on the molecule-specific base-conversion pattern in the DNA molecules;
(v) determining, from the information in Step (iv), the sequence of the RNA molecules which correspond to the DNA molecules; and
(vi) determining, from the information in Step (v), the number of copies of one or more RNA molecule in the population.

In a third aspect, the invention provides a method for determining the sequence of one or more RNA molecule in a population of RNA molecules, comprising the steps of:
(i) providing a population of RNA molecules;
(ii) subjecting the population of RNA molecules to error-prone reverse transcription, to generate a population of DNA molecules in which each DNA molecule comprises one or more base-conversion relative to the corresponding RNA molecule and wherein each DNA molecule comprises a molecule-specific base-conversion pattern;
   and using the molecule-specific base-conversion pattern to determine the sequence of the RNA molecule that corresponds to the one or more DNA molecule

In some embodiments of the third aspect, the step of using the molecule-specific base-conversion pattern to determine the number of copies of the one or more RNA molecule in a population further comprises the following, performed after Step (ii):
(iii) determining the sequence of overlapping fragments of DNA molecules in the population;
(iv) determining, from the information in step (iii), the sequence of one or more DNA molecule in the population, by assembling the sequence of overlapping fragments based on the molecule-specific base-conversion pattern of the DNA molecule; and
(v) determining, from the information in step (iv), the sequence of the RNA molecule which corresponds to the one or more DNA molecule.

In a fourth aspect, the invention provides a method for determining the sequence of one or more RNA molecule in a population of RNA molecules, comprising the steps of:
(i) providing a population of RNA molecules;
(ii) subjecting the population of RNA molecules to error-prone reverse transcription, to generate a population of DNA molecules in which each DNA molecule comprises one or more base-conversion relative to the corresponding RNA molecule and wherein each DNA molecule comprises a molecule-specific base-conversion pattern;
(iii) determining the sequence of overlapping fragments of DNA molecules in the population;
(iv) determining, from the information in step (iii), the sequence of one or more DNA molecule in the population, by assembling the sequence of overlapping fragments based on the molecule-specific base-conversion pattern of the DNA molecule; and
(v) determining, from the information in step (iv), the sequence of the RNA molecule which corresponds to the one or more DNA molecule.

By “one or more RNA molecule” we include the meaning of an RNA molecule with a unique sequence. The sequence of the one or more RNA molecule may differ from that of other RNA molecules in a population of RNA molecules because it is derived from a different gene, it is sequence variant of the same gene, an allelic variant of the same gene, a splice variant of the same gene; an RNA isoform resulting from alternative use of promoters in the same gene, an RNA isoform resulting from alternative use of splice sites in the same gene, or an RNA isoform resulting from alternative use of polyadenylation sites in the same gene.

By “population of RNA molecules” we include the meaning of a plurality of individual RNA molecules having the same or different sequences that are to be analysed using the methods disclosed herein. For example, a population of RNA molecules may contain multiple copies of the same RNA molecule; or, more typically, may contain a mixture of RNA molecules having different sequences, optionally wherein each RNA sequence is present at a different copy number. Examples of populations of RNA molecules include but are not limited to: whole RNA obtained from a single cell, multiple cells, or tissue; nuclear or cytoplasmic RNA obtained from a single cell, multiple cells, or tissue; purified pre-mRNA and/or mRNA; free RNA obtained from bodily fluids such as blood, cerebrospinal fluid, and urine; in vitro transcribed RNA; or combinations thereof. For instance, a population of RNA molecules may comprise RNA molecules derived from different sources that are analysed together as a single experiment using the methods disclosed herein.

By “population of DNA molecules” we include the meaning of a plurality of individual DNA molecules having the same or different sequences. For example, a population of DNA molecules may contain multiple copies of the same DNA molecule; or, more typically, may contain a mixture of DNA molecules having different sequences, optionally wherein each DNA sequence is present at a different copy number. In the context of the present invention such a population may be a plurality of individual cDNA molecules produced by reverse transcription of a population of RNA molecules, such as a population of RNA molecules as defined herein.

By “same sequence” we include the meaning of RNA molecules having identical sequences to one another; or DNA molecules having identical sequences to one another.

By “different sequence” we include the meaning of RNA molecules whose sequences differ from one another; or DNA molecules whose sequences differ from one another. For example, RNA molecules may have different sequences because they are produced from different genes or because they are differently processed transcripts derived from the same gene (e.g. splice variants). In the case of DNA molecules, those molecules may have different sequences because they are generated from different RNA molecules during reverse transcription or amplified from different template DNA molecules (e.g. in a PCR process), or are sequence variants of a gene or allele.

By “error-prone reverse transcription” we include the meaning of a reverse transcription process in which the resultant DNA molecules have changes in sequence relative to the template RNA molecules from which they are derived. In the context of the present invention, error-prone reverse transcription is reverse transcription that is performed in order to deliberately incorporate sequence changes into the DNA molecules produced by reverse transcription. This can be achieved in three principal manners: (i) the reverse transcriptase enzyme incorporating a base that is not complementary to the RNA template molecule in the first strand cDNA; (ii) the reverse transcriptase enzyme incorporating a non-canonical base into the first strand cDNA, thereby resulting in more frequent errors during second strand cDNA synthesis; (iii) the reverse transcriptase enzyme incorporating a non-canonical base into first strand cDNA, wherein the non-canonical base has altered susceptibility/tolerance to chemical treatment, thereby resulting in an alteration in the frequency of errors at the non-canonical base positions during second strand cDNA synthesis after exposure to such chemical treatment. In each of the three examples presented above, the double-stranded cDNA generated from the RNA template molecules include base-conversions that result from errors made during the reverse transcription process.

By “base-conversion” we include the meaning of a change in a DNA molecule produced by reverse transcription that results in a change in the base sequence of DNA molecules amplified from that DNA molecule relative to the base sequence of the corresponding RNA template molecule in the population of RNA molecules. The change in the DNA molecule may, for example, be induced through an error during reverse transcription (i.e. a misincorporation of a base not present in the template RNA molecule during first or second strand cDNA synthesis), through chemical modification of an RNA molecule prior to reverse transcription, or through chemical modification of DNA molecules after reverse transcription (but prior to amplification). The change in the DNA molecule may also, for instance, be induced through an error or the incorporation of a non-canonical base during reverse transcription (e.g. the incorporation of a base that is not a canonical complementary base to the corresponding base in the template RNA). For example, chemical modifications that deaminate cytosine (which base pairs with guanine) lead to production of uracil (which base pairs with adenine) and can induce GC-to-AT transitions. In relation to base analogues, the purine analogue 2-aminopurine is an analogue of guanine or adenine that can base pair with either thymine (as a thymine analogue) or cytosine (as a guanine analogue), and so can induce AT-to-GC or GC-to-AT transitions, whereas 5-bromouracil (5-BrU) is an analogue of thymine and can base pair with adenine (as 5-BrU keto) or guanine (as 5-BrU enol) and so can induce AT-to-GC transitions. In another example, a base analogue can be incorporated during reverse transcription so that the resulting cDNA molecules or subsequently amplified molecules contain base changes relative to the RNA template molecule.

By “base analogue” we include the meaning of a molecule that has a similar structure to one of the four canonical nitrogenous bases present in DNA (i.e. guanine, cytosine, adenine, and thymine) and can substitute for one of those canonical bases by a reverse transcriptase enzyme during cDNA synthesis or by a DNA polymerase enzyme during DNA synthesis. In the context of the present invention, a base analogue introduced into a DNA molecule produced during reverse transcription is able to form altered base-pairing with a canonical base present in an RNA molecule (i.e. guanine, cytosine, adenine, and uracil). During subsequent amplification of the DNA molecules produced by reverse transcription, the base analogue may be paired with a base that is different from the one present in the corresponding RNA molecule in the population of RNA molecules, resulting in a stable and specific base-conversion at that position in the sequence of DNA molecules amplified from that particular DNA molecule. Different base analogues form different altered base-pairings and so are able induce different base-conversions.

By “molecule-specific base-conversion pattern” we include the meaning of a pattern of base-conversions that is unique to a single, individual DNA molecule present in the population of DNA molecules produced by reverse transcription. The molecule-specific base-conversion pattern is relative to the sequence of the corresponding RNA molecule from which the DNA molecule was derived during reverse transcription and is stably propagated in sequences amplified from that DNA molecule. Accordingly, the molecule-specific base-conversion pattern can be used to identify all molecules amplified from an individual DNA molecule in the population of DNA molecules produced by reverse transcription.

It is important that the molecule-specific base-conversion pattern is stably-associated with all molecules derived from an individual DNA molecule produced by reverse transcription. For instance, if new base-conversions arise and/or alterations to existing base-conversion patterns occur during amplification and/or sequencing then molecules will arise that have new base-conversion patterns that were not present in the DNA molecules produced by reverse transcription. The production of molecules with new base-conversion patterns during amplification and/or sequencing would lead to an overestimation of the number of individual molecules of a particular sequence in the population of DNA molecules produced by reverse transcription, and, consequently, an overestimation of the number of copies of the corresponding RNA molecule in the initial population of RNA molecules.

Thus, in view of the importance of minimising and/or preventing new molecule-specific base-conversion patterns arising during the steps following reverse transcription (e.g. amplification and sequencing), the conditions that induce base-conversions during reverse transcription are removed before subsequent steps of the methods disclosed herein. For instance, the conditions that induce base-conversion during reverse transcription may be removed from the population of DNA molecules by cleaning-up and/or purifying the population of DNA molecules. For example, the conditions that induce base-conversion during reverse transcription may be removed from the population of DNA molecules by methods such as dilution, phenol chloroform extraction, bead clean-up, enzymatic removal, and/or thermal degradation.

In some embodiments of the methods disclosed herein, the methods also allow determination of the origin and strandedness of the analysed sequencing reads as being RNA transcribed from the positive strand, RNA transcribed from the negative strand (together referred to as “strandedness”), or reads that originate from DNA (for example, genomic DNA).

By “strandedness” we mean whether the sequence of the original RNA molecule is present on the positive strand or negative strand of the DNA from which it is transcribed.

Typically, reverse transcription reactions are carried out using template RNA, a reverse transcriptase enzyme, dNTPs, and primer molecules. A reverse transcription reaction may also contain relevant salts and/or other additives. Examples of commercial reverse transcriptase enzymes are known in the art and include enzymes such as AMV reverse transcriptase (New England Biolabs), SmartScribe II (Takara), Maxima H-minus (Thermofisher), RevertAid (Thermofisher), or any of the SuperScript I to IV reverse transcriptases (Thermofisher). The reverse transcriptase used may or may not have ribonuclease H activity and/or template switching ability. Concentrations of dNTPs used during reverse transcription usually range from about 0.5 to about 1 mM per dNTP. Reverse transcription can be performed with oligo-dT, random hexamer primers, or gene-specific primers. Temperatures for reverse transcription reactions can vary but are usually from of 37° C. to 55° C. The quantity of RNA that serves as the template in a typical reverse transcription reaction can range from picograms of RNA template to micrograms of RNA template. For example, the quantity of RNA template may be less than 1 picogram of RNA.

In some embodiments of the methods disclosed herein, the population of RNA molecules comprises RNA molecules with different sequences and/or RNA molecules with the same sequence.

In some embodiments of the methods disclosed herein, the population of RNA molecules analysed comprises at least 1 individual RNA molecule, 10 individual RNA molecules, 100 individual RNA molecules, at least 1,000 individual RNA molecules, at least 10,000 individual RNA molecules, at least 25,000 individual RNA molecules, at least 50,000 individual RNA molecules, at least 75,000 individual RNA molecules, at least 100,000 individual RNA molecules, at least 250,000 individual RNA molecules, at least 500,000 individual RNA molecules, at least 750,000 individual RNA molecules, at least 1,000,000 individual RNA molecules, at least 10,000,000 individual RNA molecules, at least 100,000,000 individual RNA molecules, at least 1,000,000,000 individual RNA molecules, at least 10,000,000,000 individual RNA molecules, or at least 100,000,000,000 individual RNA molecules. In a preferred embodiment, the population of RNA molecules analysed comprises at least 100,000 individual RNA molecules.

In some embodiments of the methods disclosed herein, the population of RNA molecules analysed comprises 1 to 1,000 individual RNA molecules, 1 to 10,000 individual RNA molecules, 1 to 25,000 individual RNA molecules, 1 to 50,000 individual RNA molecules, 1 to 100,000 individual RNA molecules, 1 to 250,000 individual RNA molecules, 1 to 500,000 individual RNA molecules, 1 to 750,000 individual RNA molecules, 1 to 1,000,000 individual RNA molecules, 1 to 10,000,000 individual RNA molecules, 1 to 100,000,000 individual RNA molecules, 1 to 1,000,000,000 individual RNA molecules, 1 to 10,000,000,000 individual RNA molecules, or 1 to 100,000,000,000 individual RNA molecules. Preferably the population of RNA molecules analysed comprises 100 to 1,000,000,000,000 individual RNA molecules, more preferably 1,000 to 1,000,000,000 individual RNA molecules, most preferably 100,000 to 100,000,000 individual RNA molecules.

In some embodiments of the methods disclosed herein, the one or more RNA molecule is present in the population of RNA molecules at a copy number of 1 to 10 copies, 1 to 20 copies, 1 to 30 copies, 1 to 40 copies, 1 to 50 copies, 1 to 60 copies, 1 to 70 copies, 1 to 80 copies, 1 to 90 copies, 1 to 100 copies, 1 to 125 copies, 1 to 150 copies, 1 to 175 copies, 1 to 200 copies, 1 to 225 copies, 1 to 250 copies, 1 to 275 copies, 1 to 300 copies, 1 to 400 copies, 1 to 500 copies, 1 to 600 copies, 1 to 700 copies, 1 to 800 copies, 1 to 900 copies, 1 to 1,000 copies, 1 to 2,000 copies, 1 to 3,000 copies, 1 to 3,000 copies, 1 to 4,000 copies, 1 to 5,000 copies, 1 to 10,000 copies, 1 to 25,000 copies, 1 to 50,000 copies, 1 to 75,000 copies, 1 to 100,000 copies, 1 to 200,000 copies, 1 to 300,000 copies, 1 to 400,000 copies, 1 to 500,000 copies, or 500,000 or more copies. Preferably the one or more RNA molecule is present in the population of RNA molecules at a copy number of 1 to 500,000 copies, more preferably 1 to 250,000 copies, yet more preferably 1 to 100,000 copies, yet more preferably 1 to 50,000 copies, most preferably 1 to 5,000 copies.

In some embodiments of the methods disclosed herein, the size range of the RNA molecules in population is 100 base pairs to 1,000 base pairs, 100 base pairs to 2,000 base pairs, 100 base pairs to 3,000 base pairs, 100 base pairs to 4,000 base pairs, 100 base pairs to 5,000 base pairs, 100 base pairs to 6,000 base pairs, 100 base pairs to 7,000 base pairs, 100 base pairs to 8,000 base pairs, 100 base pairs to 9,000 base pairs, 100 base pairs to 10,000 base pairs, 100 base pairs to 11,000 base pairs, 100 base pairs to 12,000 base pairs, 100 base pairs to 13,000 base pairs, 100 base pairs to 14,000 base pairs, 100 base pairs to 15,000 base pairs, 100 base pairs to 16,000 base pairs, 100 base pairs to 17,000 base pairs, 100 base pairs to 18,000 base pairs, 100 base pairs to 19,000 base pairs, 100 base pairs to 20,000 base pairs, 500 base pairs to 20,000 base pairs, 1,000 base pairs to 20,000 base pairs, or 2,000 base pairs to 20,000 base pairs.

In some embodiments of the methods disclosed herein, the population of RNA molecules may be from a single cell, a plurality or population of cells, tissue, or a bodily fluid such as blood, cerebrospinal fluid, or urine. In some embodiments, the population of RNA molecules is from viral particles.

The population of RNA molecules may be from any cell. In some embodiments, the cell is a eukaryotic cell (e.g. from a metazoan, a plant, or a fungus), bacterial cells (i.e. from Eubacteria), or archaeal cells (i.e. from Archaebacteria). In some embodiments, the population of RNA molecules is from a subcellular compartment of a cell. For example, in eukaryotic cells, the population of RNA molecules may be from compartments such as the nucleus, cytoplasm, mitochondrion, or chloroplast.

In some embodiments of the methods disclosed herein, the population of RNA molecules comprises one or more RNA molecule selected from the group consisting of: messenger RNA (mRNA), precursor mRNA (pre-mRNA), antisense RNA (asRNA) and precursors thereof, enhancer RNA and precursors thereof, long non-coding RNA (lncRNA) and precursors thereof, microRNA (miRNA) and precursors thereof, ribosomal RNA (rRNA) and precursors thereof, transfer RNA (tRNA) and precursors thereof, histone RNA and precursors thereof, small nucleolar RNA (snoRNA) and precursors thereof, small nuclear RNAs (snRNA) and precursors thereof, mitochondrial RNA and precursors thereof, viral RNA, transposon RNA, synthetic RNA, in vitro transcribed RNA, or combinations thereof.

In some embodiments of the methods disclosed herein, prior to Step (i), the population of RNA molecules is purified and/or enriched for particular classes of RNA molecule. For example, the population of RNA molecules may be enriched for pre-mRNA and/or mRNA molecules.

In some embodiments of the methods disclosed herein, Step (ii) comprises introducing one or more base-conversion into each DNA molecule at a total rate of about 0.5% to about 99.5%, about 2% to about 98%, about 3% to about 97%, about 4% to about 96%, about 5% to about 95%, about 6% to about 94%, about 7% to about 93%, about 8% to about 92%, about 9% to about 91%, about 10% to about 90%, about 11% to about 89%, about 12% to about 88%, about 13% to about 87%, about 14% to about 86%, about 15% to about 85%, about 16% to about 84%, about 17% to about 83%, about 18% to about 82%, or about 19% to about 81%, about 20% to about 80%, about 25% to about 75%, about 30% to about 70%, about 35% to about 65%, about 40% to about 60%, about 45% to about 55%, about 50% to about 99.5%, about 55% to about 99.5%, about 60% to about 99.5%, about 65% to about 99.5%, about 70% to about 99.5%, about 75% to about 99.5%, about 80% to about 99.5%, about 81% to about 99.5%, about 82% to about 99.5%, about 83% to about 99.5%, about 84% to about 99.5%, about 85% to about 99.5%, about 86% to about 99.5%, about 87% to about 99.5%, about 88% to about 99.5%, about 89% to about 99.5%, about 90% to about 99.5%, about 91% to about 99.5%, about 92% to about 99.5%, about 93% to about 99.5%, about 94% to about 99.5%, about 95% to about 99.5%, about 96% to about 99.5%, about 97% to about 99.5%, about 98% to about 99.5%, or about 99% to about 99.5%. Preferably Step (ii) comprises introducing one or more base-conversion into each DNA molecule at a total rate of about 0.5% to about 99.5%, more preferably at a rate of about 2% to about 98%, yet more preferably about 5% to about 95%, yet more preferably about 5% to about 50%, yet more preferably about 5% to about 20%. Most preferably, Step (ii) comprises introducing one or more base-conversion into each DNA molecule at a total rate of about 15% to about 30%.

In some embodiments of the methods disclosed herein, Step (ii) comprises introducing one or more base-conversion into each DNA molecule at a total rate of at least 0.5%, 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, or at least 50%. Preferably Step (ii) comprises introducing one or more base-conversion into each DNA molecule at a total rate of at least 0.5%, more preferably at least 1%, yet more preferably at least 3%, yet more preferably at least 5%. Most preferably, Step (ii) comprises introducing one or more base-conversion into each DNA molecule at a total rate of at least 15%.

The rate of base-conversion per molecule is measured as a percentage of the total sequenced bases that have been converted in an individual DNA molecule produced by reverse transcription (and its amplified descendant DNA molecules) relative to the corresponding RNA molecule in the initial population of RNA molecules. For example, rate of base-conversion is often used in terms of a percentage conversion per eligible base. For example, a 50% C-to-T conversion would indicate that 50% of cytosines are converted to thymines.

In some embodiments of the methods disclosed herein, Step (ii) comprises reverse transcription in the presence of one or more base analogue.

In preferred embodiments of the methods disclosed herein, the one or more base analogue is selected from the group consisting of:

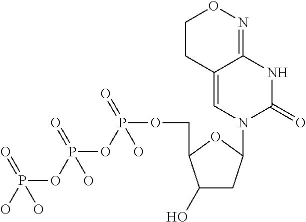 [SMILES: O=C1NC2=NOCCC2=CN1C1CC(O)C(COP(=O)(O)OP(=O)(O)OP(=O)(O)O)O1]

2′-deoxy-P-nucleoside-5′-triphosphate (dPTP) (TriLink: N-2037; Jena Bioscience: NU-1119)

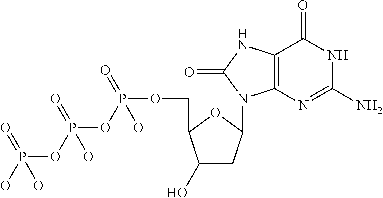 [SMILES: Nc1nc2c([nH]c(=O)n2C2CC(O)C(COP(=O)(O)OP(=O)(O)OP(=O)(O)O)O2)c(=O)[nH]1]

8-Oxo-2′-deoxyguanosine-5′-triphosphate (8-oxo-GTP) (Trilink: N-2034);

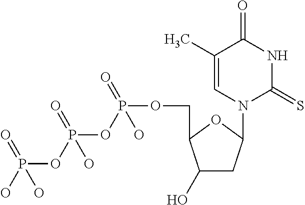 [SMILES: Cc1cn(C2CC(O)C(COP(=O)(O)OP(=O)(O)OP(=O)(O)O)O2)c(=S)[nH]c1=O]

2-Thiothymidine-5′-triphosphate (2-thioTTP) (TriLink: N-2035);

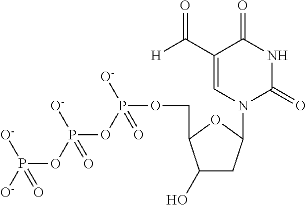 [SMILES: O=Cc1cn(C2CC(O)C(COP(=O)([O-])OP(=O)([O-])OP(=O)([O-])[O-])O2)c(=O)[nH]c1=O]

5-Formyl-2′-deoxyuridine-5′-triphosphate (TriLink: N-2067)

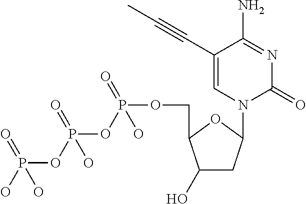 [SMILES: CC#Cc1cn(C2CC(O)C(COP(=O)(O)OP(=O)(O)OP(=O)(O)O)O2)c(=O)nc1N]

5-Propynyl-2′-deoxycytidine-5′-Triphosphate (TriLink: N-2016)

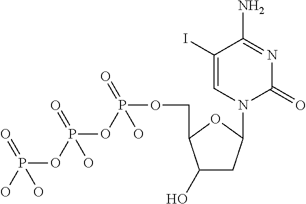 [SMILES: Nc1nc(=O)n(C2CC(O)C(COP(=O)(O)OP(=O)(O)OP(=O)(O)O)O2)cc1I]

5-Iodo-2′-deoxycytidine-5′-triphosphate (TriLink: N-2023)

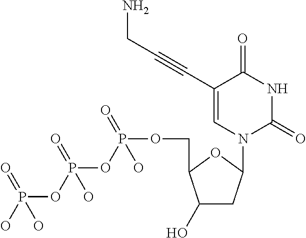 [SMILES: NCC#Cc1cn(C2CC(O)C(COP(=O)(O)OP(=O)(O)OP(=O)(O)O)O2)c(=O)[nH]c1=O]

5-Propargylamino-2′-deoxyuridine-5′-triphosphate (N-2062)
   or combinations thereof.

In some embodiments of the methods disclosed herein, Step (ii) comprises reverse transcription in the presence of a sub-optimal amount of one or more dNTP base.

By “sub-optimal amount of one or more dNTP base” we include the meaning of a dNTP base at a concentration that is lower than the concentration typically used in a reverse transcription reaction. Reverse transcription reactions commonly contain dNTPs at concentrations in the range of 0.2 mM to 0.5 mM. It is also possible to use higher concentrations of dNTPs (e.g. 0.5 mM to 1 mM) in reverse transcription reactions. By “sub-optimal amount of one or more dNTP base” we also include the meaning of a dNTP base having a concentration that is different (i.e. lower or higher) relative to one or more of the other dNTPs in a reaction mix. It will be appreciated that performing reverse transcription in the presence of a base analogue and with a sub-optimal amount of one or more dNTP base can result in the incorporation of errors into the sequence of the resultant DNA molecules.

In some embodiments of the methods disclosed herein, Step (ii) comprises reverse transcription in the presence of one or more dNTP bases at a concentration of less than 0.5 mM, less than 0.4 mM, less than 0.3 mM, less than 0.2 mM, or less than 0.1 mM. Preferably Step (ii) comprises reverse transcription in the presence of one or more dNTP bases at a concentration of less than 0.3 mM, more preferably less than 0.2 mM, most preferably less than 0.1 mM.

In some embodiments of the methods disclosed herein, Step (ii) comprises reverse transcription in the presence of one or more dNTP bases at a concentration of at least 0.1 mM, at least 0.2 mM, at least 0.3 mM, at least 0.4 mM, at least 0.5 mM, at least 0.6 mM, at least 0.7 mM, at least 0.8 mM, at least 0.9 mM, at least 1 mM, at least 1.1 mM, at least 1.2 mM, at least 1.3 mM, at least 1.4 mM, or at least 1.5 mM. Preferably Step (ii) comprises reverse transcription in the presence of one or more dNTP bases at a concentration of at least 0.5 mM, more preferably at least 1 mM, most preferably at least 1.5 mM.

In some embodiments, the method comprises incorporating one or more base analogue into the one or more RNA molecule in the population of RNA molecules prior to Step (i). In some embodiments, the one or more base analogue is 4-thio-uridine.

In some embodiments of the methods disclosed herein, Step (ii) further comprises the step of chemically-modifying the population of RNA molecules, prior to subjecting the population of RNA molecules to reverse transcription. It will be appreciated that such chemical-modification can result in the incorporation of errors into the sequence of the resultant DNA molecules. It is also possible to edit the population of RNA molecules with editing enzymes such as APOBEC1, which is able to deaminate RNA cytosines that result in C-to-T edits (Grünewald et al, 2019. Nature, 569:433-437), prior to subjecting the population of RNA molecules to reverse transcription. Another possibility is to incorporate a base analogue such as 4-thio-uridine into RNAs during transcription of those molecules. For instance, compounds such as 4-thio-uridine can be incorporated through cellular transcription by introducing them to cell media during culturing. Alternatively, such compounds can be incorporated during in vitro transcription as part of the process of sequencing library preparation, for example, as used in CEL-seq and CEL-seq2 (Hashimshony et al, 2012. Cell Rep., 2 (3): 666-73; Hashimshony et al, 2016. Genome Biol., 17:77). By incorporating 4-thio-uridine into RNA, a target is provided for subsequent chemical modification by alkylation with iodoacetamide (Herzog et al, 2017. Nat. Methods, 14 (12): 1198-1204). Alternatively, 4-thio-uridine-containing RNA can be subjected to oxidative nucleophilic aromatic substitution using for example the oxidants NaIO3 or mCPBA and the nucleophile 2,2,2-trifluoroethylamine (Schofield et al, 2018. Nat. Methods 15, 221-225). These different modifications of 4-thio-uridine bases are analogous to cytosine, resulting in incorporation of guanidine instead of adenosine during reverse transcription and the creation of unique patterns of errors or base-conversions in the cDNA derived from each RNA molecule. After amplification, such patterns can be used to identify the molecule-of-origin for short reads corresponding to parts of these RNA molecules.

By “chemically-modifying” we include the meaning of a process that alters the chemical constitution and/or structure of an RNA molecule or a DNA molecule. In particular, in the context of the present application, chemical modification relates to treatments that lead to alterations in the chemical constitution and/or structure of the nitrogenous base components of an RNA molecule or DNA molecule. The frequency of base-conversions can be tuned by the incorporation during reverse transcription of a non-canonical base with altered susceptibility/tolerance to chemical modification.

In some embodiments, the step of chemically-modifying the population of RNA molecules comprises alkylating the population of RNA molecules. In some embodiments, the alkylating is by iodoacetamide treatment or oxidative nucleophilic aromatic substitution.

In some embodiments of the methods disclosed herein, Step (ii) further comprises the sub-step of chemically-modifying the population of DNA molecules generated by reverse transcription.

In some embodiments, the chemical modification of the population of DNA molecules generated by reverse transcription comprises a deamination reaction. In certain embodiments, the deamination is carried out using one or more selected from the list consisting of: bisulfite treatment, the reduction of (previously modified) nucleosides with pyridine borane or its derivative 2-picoline-borane (Liu Y. et al. 2019. Nature Biotechnology 37:424-429), or using enzymatic deamination strategies such as, for example, APOBEC treatment.

In the case of bisulfite treatment of DNA produced by reverse transcription, that treatment results in the conversion of unmethylated cytosines to uracil, while methylated cytosines are unaffected. Accordingly, by incorporating methylated cytosines at a given percentage during reverse transcription, and then performing bisulfite treatment it is possible to obtain partially converted libraries with a high percentage of C-to-T base-conversions.

In some embodiments of the methods disclosed herein, Step (ii) comprises reverse transcription using an error-prone reverse transcriptase enzyme.

By “error-prone reverse transcriptase enzyme” we include the meaning of a reverse transcriptase enzyme that introduces base-conversions in the complementary strand of the DNA molecules it produces by reverse transcription relative to the RNA template sequence.

In some embodiments of the methods disclosed herein, the error-prone reverse transcriptase has an error rate of at least 1 error per 100 bases, at least 2 errors per 100 bases, at least 3 errors per 100 bases, at least 4 errors per 100 bases, at least 5 errors per 100 bases, at least 6 errors per 100 bases, at least 7 errors per 100 bases, at least 8 errors per 100 bases, at least 9 errors per 100 bases, at least 10 error per 100 bases, at least 11 errors per 100 bases, at least 12 errors per 100 bases, at least 13 errors per 100 bases, at least 14 errors per 100 bases, at least 15 errors per 100 bases, at least 16 errors per 100 bases, at least 17 errors per 100 bases, at least 18 errors per 100 bases, at least 19 errors per 100 bases, at least 20 errors per 100 bases, at least 25 errors per 100 bases, at least 30 errors per 100 bases, at least 35 errors per 100 bases, at least 40 errors per 100 bases, at least 45 errors per 100 bases, at least 50 errors per 100 bases, at least 55 errors per 100 bases, or at least 60 errors per 100 bases.

An error-prone reverse transcriptase enzyme can be produced using approaches known in the art of molecular biology and protein engineering. The most commonly used strategies for protein engineering are rational protein design (i.e. using knowledge of the function and/or sequence of a protein to make defined amino acid changes) and directed evolution (i.e. using rounds of random mutagenesis and selection on the basis of a desired characteristic), and a combination of each approach is often used by researchers. A modified reverse transcription enzyme with increased to incorporating modified bases can also be produced using approaches known in the art of molecular biology and protein engineering (see, for example, Zhou et al, 2019. Nat. Methods, 16, 1281-1288).

In some embodiments of the methods disclosed herein, Step (iii) comprises the step of amplifying the population of DNA molecules from Step (ii) to generate one or more amplicon of each DNA molecule in the population.

By “amplicon” we include the meaning of a DNA molecule that has been amplified from a DNA template, for example, a PCR product.

In some embodiments of the methods disclosed herein, the step of amplifying the population of DNA molecules comprises high-fidelity amplification.

In some embodiments of the methods disclosed herein, the step of amplifying the population of DNA molecules comprises PCR amplification.

By “high fidelity amplification” we include the meaning of amplification that results in amplicons that have very few or no sequence changes relative to the corresponding sequence in the original template molecule (e.g. the original cDNA molecule). Such high fidelity amplification may be carried out using a commercial proof-reading DNA polymerase enzyme.

In some embodiments of the methods disclosed herein, a non-proof-reading DNA polymerase enzyme is used during second strand cDNA synthesis, and then a high fidelity, proof-reading DNA polymerase enzyme is used for the step of amplifying the population of DNA molecules. A non-proof-reading DNA polymerase enzyme (e.g. Taq DNA polymerase) is preferred for second strand cDNA synthesis because it is more likely to tolerate the presence of a non-canonical base in the cDNA first strand and therefore introduce a base-conversion in the cDNA second strand. A proof-reading DNA polymerase is preferred for the step of amplifying the population of DNA molecules because it is more likely to maintain the base-conversion patterns introduced during the error-prone reverse transcription step.

In some embodiments of the methods disclosed herein, the step of amplifying the population of DNA molecules is performed in the absence of a base analogue.

In some embodiments of the methods disclosed herein, at least the first cycle of the step of amplifying the population of DNA molecules is performed in the presence of a sub-optimal amount of one or more dNTP base.

In some embodiments, at least the first cycle of the step of amplifying the population of DNA molecules is performed in the presence of one or more dNTP bases at a concentration of less than 0.5 mM, less than 0.4 mM, less than 0.3 mM, less than 0.2 mM, or less than 0.1 mM. Preferably, at least the first cycle of the step of amplifying the population of DNA molecules is performed in the presence of one or more dNTP bases at a concentration of less than 0.3 mM, more preferably less than 0.2 mM, most preferably less than 0.1 mM.

In some embodiments of the methods disclosed herein, at least the first cycle of the step of amplifying the population of DNA molecules is performed in the presence of one or more dNTP bases at a concentration of at least 0.1 mM, at least 0.2 mM, at least 0.3 mM, at least 0.4 mM, at least 0.5 mM, at least 0.6 mM, at least 0.7 mM, at least 0.8 mM, at least 0.9 mM, at least 1 mM, at least 1.1 mM, at least 1.2 mM, at least 1.3 mM, at least 1.4 mM, or at least 1.5 mM. Preferably, at least the first cycle of the step of amplifying the population of DNA molecules is performed in the presence of one or more dNTP bases at a concentration of at least 0.5 mM, more preferably at least 1 mM, most preferably at least 1.5 mM.

After incorporation of one or more base analogues in the first-strand cDNA, varying amounts of individual dNTPs (e.g. sub-optimal or uneven amounts of one or more dNTP) can be used in the amplification first cycle. In that cycle the first-strand cDNA serves as a template for amplification, and by varying the amount of dNTPs relative to one another in the reaction it is possible to bias a base analogue in the first-strand cDNA towards preferentially pairing with one base over other bases, thereby influencing the identity of a conversion event and/or altering overall conversion rate at sites in the first-strand cDNA having a base analogue.

It will be appreciated that the use of sub-optimal amounts of one or more dNTP base during at least the first cycle of an amplification step is applicable to amplification of any polynucleotide comprising one or more base analogues and can similarly bias such base analogues towards preferentially pairing with one base over other bases.

Accordingly, a further aspect of the invention relates to a method for generating base-conversions in one or more polynucleotide molecule in a population of polynucleotide molecules, comprising the steps of:
(i) providing a population of polynucleotide molecules, wherein one or more of the polynucleotide molecules comprises one or more base analogue;
(ii) amplifying the population of polynucleotide molecules from Step (i) to generate one or more amplicon of each polynucleotide molecule in the population, wherein at least the first cycle of the step of amplifying is performed in the presence of a sub-optimal amount of one or more dNTP base.

In some embodiments of the method for generating base-conversions in one or more polynucleotide molecule in a population of polynucleotide molecules, the one or more polynucleotide molecule is a cDNA molecule, a DNA molecule, or an RNA molecule (including a double-stranded RNA molecule).

In some embodiments of the method for generating base-conversions in one or more polynucleotide molecule in a population of polynucleotide molecules, at least the first cycle of the step of amplifying the population of polynucleotide molecules is performed in the presence of one or more dNTP bases at a concentration of less than 0.5 mM, less than 0.4 mM, less than 0.3 mM, less than 0.2 mM, or less than 0.1 mM. Preferably, at least the first cycle of the step of amplifying the population of polynucleotide molecules is performed in the presence of one or more dNTP bases at a concentration of less than 0.3 mM, more preferably less than 0.2 mM, most preferably less than 0.1 mM.

In some embodiments of the method for generating base-conversions in one or more polynucleotide molecule in a population of polynucleotide molecules, at least the first cycle of the step of amplifying the population of polynucleotide molecules is performed in the presence of one or more dNTP bases at a concentration of at least 0.1 mM, at least 0.2 mM, at least 0.3 mM, at least 0.4 mM, at least 0.5 mM, at least 0.6 mM, at least 0.7 mM, at least 0.8 mM, at least 0.9 mM, at least 1 mM, at least 1.1 mM, at least 1.2 mM, at least 1.3 mM, at least 1.4 mM, or at least 1.5 mM. Preferably, at least the first cycle of the step of amplifying the population of polynucleotide molecules is performed in the presence of one or more dNTP bases at a concentration of at least 0.5 mM, more preferably at least 1 mM, most preferably at least 1.5 mM.

In some embodiments of the method for generating base-conversions in one or more polynucleotide molecule in a population of polynucleotide molecules, the step of amplifying the population of polynucleotide molecules comprises high-fidelity amplification.

In some embodiments of the method for generating base-conversions in one or more polynucleotide molecule in a population of polynucleotide molecules, the step of amplifying the population of polynucleotide molecules comprises PCR amplification.

In some embodiments of the method for generating base-conversions in one or more polynucleotide molecule in a population of polynucleotide molecules, the step of amplifying the population of polynucleotide molecules is performed in the absence of a base analogue.

In embodiments wherein reverse transcription is carried out in the presence of one or more base analogue, wherein chemical modification is carried out on a population of RNA molecules prior to reverse transcription, or wherein chemical modification is carried on a population of DNA molecules produced by reverse transcription, the conditions or treatments that induce base-conversions are removed prior to the amplification step. For example, where a base analogue has been used in the reverse transcription step, any unincorporated base analogue molecules are removed (or degraded) prior to amplification by methods such as dilution, phenol chloroform extraction, bead clean-up, enzymatic removal, and/or thermal degradation.

In some embodiments of the methods disclosed herein, Step (iii) comprises the step of fragmenting the population of DNA molecules and/or the one or more amplicon of each DNA molecule in the population, to generate overlapping fragments. In some embodiments, the population of DNA molecules and/or the one or more amplicon of each DNA molecule in the population are purified prior to fragmentation.

In some embodiments of the methods disclosed herein, the step of fragmenting the population of DNA molecules and/or the one or more amplicon of each DNA molecule in the population comprises tagmentation, DNA shearing, and/or enzymatic fragmentation.

By “tagmentation” we include the meaning of a process for the integration of sequencing adapters into DNA using a transposase, for example, integration of partial sequencing adapters.

In some embodiments of the methods disclosed herein, the fragments are about 50 base pairs to about 2000 base pairs in length, about 50 base pairs to about 1900 base pairs in length, about 50 base pairs to about 1800 base pairs in length, about 50 base pairs to about 1700 base pairs in length, about 50 base pairs to about 1600 base pairs in length, about 50 base pairs to about 1500 base pairs in length, about 50 base pairs to about 1400 base pairs in length, about 50 base pairs to about 1300 base pairs in length, about 50 base pairs to about 1200 base pairs in length, about 50 base pairs to about 1100 base pairs in length, about 50 base pairs to about 1000 base pairs in length, about 50 base pairs to about 950 base pairs in length, about 50 base pairs to about 900 base pairs in length, about 50 base pairs to about 850 base pairs in length, about 50 base pairs to about 800 base pairs in length, about 50 base pairs to about 750 base pairs in length, about 50 base pairs to about 700 base pairs in length, about 50 base pairs to about 650 base pairs in length, about 50 base pairs to about 600 base pairs in length, about 50 base pairs to about 550 base pairs in length, about 50 base pairs to about 500 base pairs in length, about 50 base pairs to about 450 base pairs in length, about 50 base pairs to about 400 base pairs in length, about 50 base pairs to about 350 base pairs in length, about 50 base pairs to about 300 base pairs in length, about 50 base pairs to about 250 base pairs in length, about 50 base pairs to about 200 base pairs in length, about 50 base pairs to about 150 base pairs in length, about 50 base pairs to about 100 base pairs in length, about 100 base pairs to about 1500 base pairs in length, about 150 base pairs to about 1400 base pairs in length, about 200 base pairs to about 1300 base pairs in length, about 250 base pairs to about 1200 base pairs in length, about 300 base pairs to about 1100 base pairs in length, about 350 base pairs to about 1000 base pairs in length, about 400 base pairs to about 1000 base pairs in length, about 450 base pairs to about 950 base pairs in length, about 500 base pairs to about 900 base pairs in length, about 550 base pairs to about 850 base pairs in length, about 600 base pairs to about 800 base pairs in length, about 650 base pairs to about 750 base pairs in length, about 700 base pairs to about 1500 base pairs in length, about 750 base pairs to about 1500 base pairs in length, about 800 base pairs to about 1500 base pairs in length, about 850 base pairs to about 1500 base pairs in length, about 900 base pairs to about 1500 base pairs in length, about 950 base pairs to about 1500 base pairs in length, about 1000 base pairs to about 1500 base pairs in length, about 1100 base pairs to about 1500 base pairs in length, about 1200 base pairs to about 1500 base pairs in length, about 1300 base pairs to about 1500 base pairs in length, or about 1400 base pairs to about 1500 base pairs in length. Preferably the fragments are about 50 base pairs to about 1500 base pairs in length, more preferably 50 base pairs to 1200 base pairs in length, yet more preferably 50 base pairs to 1000 base pairs in length, most preferably 50 base pairs to 800 base pairs in length.

By “overlapping fragments” we include the meaning of any overlapping parts of at least two DNA sequences. The sequences that contain overlapping parts may be from those obtained directly from a short-read sequencing experiment (i.e. as single-end or paired-end reads) or from partially reconstructed DNA sequences. Partial reconstruction of DNA sequences can be achieved using, for example, molecular barcodes, or in iterative fashion using the methods disclosed herein.

In some embodiments of the methods disclosed herein, the length of overlapping sequence required to identify and assemble overlapping fragments with the same molecule-specific base-conversion pattern is at least 10 base pairs, at least 15 base pairs, at least 20 base pairs, at least 25 base pairs, at least 30 base pairs, at least 35 base pairs, at least 40 base pairs, at least 45 base pairs, at least 50 base pairs, at least 55 base pairs, at least 60 base pairs, at least 65 base pairs, at least 70 base pairs, at least 75 base pairs, at least 80 base pairs, at least 85 base pairs, at least 90 base pairs, at least 95 base pairs, at least 100 base pairs, at least 125 base pairs, at least 150 base pairs, at least 175 base pairs, or at least 200 base pairs. Preferably the length of overlapping sequence required to identify and assemble overlapping fragments with the same molecule-specific base-conversion pattern is at least 200 base pairs, more preferably at least 100 base pairs, yet more preferably at least 75 base pairs, most preferably at least 50 base pairs.

In some embodiments of the methods disclosed herein, the length of overlapping sequence required to identify and assemble overlapping fragments with the same molecule-specific base-conversion pattern is less than 500 base pairs, less than 450 base pairs, less than 400 base pairs, less than 350 base pairs, less than 300 base pairs, less than 250 base pairs, less than 200 base pairs, less than 175 base pairs, less than 150 base pairs, less than 125 base pairs, less than 100 base pairs, less than 95 base pairs, less than 90 base pairs, less than 85 base pairs, less than 80 base pairs, less than 75 base pairs, less than 70 base pairs, less than 65 base pairs, less than 60 base pairs, less than 55 base pairs, less than 50 base pairs, less than 45 base pairs, less than 40 base pairs, less than 35 base pairs, less than 30 base pairs, less than 25 base pairs, less than 20 base pairs, less than 15 base pairs, or less than 10 base pairs. Preferably the length of overlapping sequence required to identify and assemble overlapping fragments with the same molecule-specific base-conversion pattern is less than 500 bases, more preferably less than 300 bases, yet more preferably less than 200 base pairs, most preferably less than 100 base pairs.

In some embodiments of the methods disclosed herein, the length of the overlapping sequence required to identify and assemble overlapping fragments with the same molecule-specific base-conversion pattern is 10 base pairs to 500 base pairs in length, 15 base pairs to 450 base pairs, 20 base pairs to 400 base pairs in length, 25 base pairs to 350 base pairs in length, 30 base pairs to 300 base pairs, 35 base pairs to 250 base pairs in length, 40 base pairs to 200 base pairs in length, 45 base pairs to 175 base pairs, 50 base pairs to 150 base pairs in length, 55 base pairs to 125 base pairs in length, 60 base pairs to 100 base pairs, 65 base pairs to 95 base pairs in length, 70 base pairs to 90 base pairs in length, 75 base pairs to 90 base pairs in length, 80 base pairs to 85 base pairs in length, 90 base pairs to 500 base pairs in length, 95 base pairs to 500 base pairs in length, 100 base pairs to 500 base pairs in length, 125 base pairs to 500 base pairs in length, 150 base pairs to 500 base pairs in length, 175 base pairs to 500 base pairs in length, 200 base pairs to 500 base pairs in length, 250 base pairs to 500 base pairs in length, 300 base pairs to 500 base pairs in length, 350 base pairs to 500 base pairs in length, 400 base pairs to 500 base pairs in length, or 450 base pairs to 500 base pairs in length. Preferably the length of overlapping sequence required to identify and assemble overlapping fragments with the same molecule-specific base-conversion pattern is 10 base pairs to 500 base pairs in length, more preferably 25 base pairs to 250 base pairs in length, yet more preferably 50 base pairs 150 base pairs in length, most preferably 50 base pairs to 100 base pairs in length.

In some embodiments of the methods disclosed herein, Step (iii) comprises sequencing overlapping fragments of the population of DNA molecules and/or the one or more amplicon of each DNA molecule in the population. In some embodiments, the population of DNA molecules and/or the one or more amplicon of each DNA molecule in the population are purified prior to sequencing.

In some embodiments, the population of DNA molecules and/or the one or more amplicon of each DNA molecule in the population is purified prior to fragmentation and/or sequencing.

Where fragmentation is performed, indexing and library amplification or PCR-free ligation is carried out. In the context of the methods described herein, indexing involves the addition of specific molecular sample barcodes to sequencing libraries derived from a particular population of RNA molecules. Such sample indexing allows multiple libraries derived from different starting populations of RNA molecules to be sequenced in parallel (for example on a flow cell), and then subsequently be used to associate the sequence reads to the correct population of RNA molecules. A sample barcode can be added to an oligo-dT primer or template-switching oligo and is therefore present at the end of a cDNA molecule produced using such oligos. With such strategies, only the subset of paired-end sequence reads that cover the 5′ or 3′ end of a molecule would have the cell/sample barcode and internal read pairs would not have a barcode. Alternatively, sample barcodes can be added after tagmentation (e.g. in the post-tagmentation PCR oligos), which leads to all sequences in the library having the barcode (i.e. so both 5′ and 3′ end fragments and internal fragments have the barcode).

By “molecular barcode” we include the meaning of a pool of nucleic acid sequences that is added to a particular population of RNA or DNA molecules and can act as a unique identifier allowing the grouping of amplified DNA sequences derived from the same initial RNA or DNA molecule. Molecular barcodes are added prior to cDNA amplification and they are typically included in the template switching oligo or oligo-dT. Molecular barcodes can also be referred to as Unique Molecular Identifiers (UMIs), and they are often a stretch of 4 to 25 random nucleotides.

Using libraries where all paired end reads have sample barcodes can aid reconstruction of the sequence of the RNA molecules in the population of RNA molecules because the search space for finding unique base-conversion patterns is smaller. However, it is still possible to reconstruct RNA sequences effectively using libraries without sample barcodes on the internal paired end reads. In the present invention, molecular barcodes are not needed since the base-conversion patterns introduced in the error-prone reverse transcription step is superior to traditional UMIs. Thus, the methods disclosed herein can be carried out using libraries where no molecules have molecular barcodes added, where a subset of molecules have molecular barcodes added, or where all molecules have molecular barcodes added. Furthermore, the methods disclosed herein can be carried out using libraries where no molecules have sample barcodes added, where a subset of molecules have sample barcodes added, or where all molecules have sample barcodes added.

In some embodiments of the methods disclosed herein, sequencing comprises a short-read sequencing method.

By “short-read sequencing method” we include the meaning of a sequencing method that does not cover the entirety of the sequenced molecules in a single sequencing read. Short-read sequencing typically generates sequencing reads with a length or about 50 base pairs to about 400 base pairs.

In some embodiments, the short-read sequencing method is selected from the list consisting of: massive parallel short-read sequencing; DNA nanoball sequencing; Illumina dye Sequencing (Solexa sequencing); 454 pyrosequencing; SOLID sequencing; Helicos single molecule fluorescent sequencing; combinatorial probe anchor synthesis (cPAS); polony sequencing; electrical sequencing chips (e.g. GenapSys); or combinations thereof.

In some embodiments of the methods disclosed herein, Step (iv) comprises:
(a) assigning overlapping fragments to an RNA molecule present in the population of RNA molecules based on their alignment to some or all of the sequence of that RNA molecule; and/or,
(b) sorting the assigned fragments based on the position in the RNA molecule at which those fragments align.

The number of overlapping DNA fragments (and their respective lengths) required to obtain sequence reads covering the whole length of an RNA present in the initial population of RNA molecules is dependent on the sequencing strategy used. Typically, as the average length of the reads generated increases, the probability of obtaining longer overlaps increases, and vice versa. Thus, there is an interplay between the sequence depth and the short-read sequencing strategy used and the evenness of the read-pairs obtained over the length of the sequence of a given RNA molecule in the initial population of RNA molecules. That interplay ultimately dictates the number of paired-end reads required to assemble the sequence of a particular RNA molecule.

Assignment and alignment of overlapping sequence fragments to an RNA molecule and the sorting of those fragments based on the position of their alignment to that RNA molecule can be carried using computational methods. For example, software can be used to map all sequence reads obtained to a database of reference sequences and then annotate each sequence read (or read-pair) based on the population DNA molecules from which that read/read-pair is derived, using, for example, molecular barcodes/UMIs present in the read/read-pairs. The annotated groups of sequenced fragments obtained through alignment to the reference sequences can then be sorted by the software based on their mapping positions on the reference sequence. Next, the position of each base-conversion in the aligned fragments is determined before probabilistic approaches are used to estimate the co-occurrence strength of pairs of base-conversions. Based on the co-occurrence information it is possible to identify groups of fragments that share the same base-conversion patterns in a statistically significant manner. The analysis is then repeated until it is no longer possible to assemble any further reads.

By “reference sequence” we include the meaning of a known sequence, typically from a database, against which sequence reads can be compared and aligned. The reference sequence may or may not be part of a reference genome.

In some embodiments of the methods disclosed herein, Step (v) comprises comparing the sequence information in Step (iv) to a reference sequence and identifying mismatches corresponding to one or more base-conversion.

Alignment software can be used to identify the correct alignment position of a short read towards the reference sequence despite the presence of many base-conversions. Examples of such software include:
STAR (https://github.com/alexdobin/STAR);
BWA (https://github.com/lh3/bwa); and
Bowtie (http://bowtie-bio.sourceforge.net/index.shtml).

Once alignment of the sequence reads has been carried out, base-conversions are spotted based on mismatches relative to the reference sequence. Again, software can be used to “spot” induced base-conversions. Such software is also able to distinguish reverse transcription induced base-conversions from mismatches that arise from mutations in the RNA molecule in the population of RNA molecules, single nucleotide polymorphisms (SNPs), and PCR/sequencing errors. This is possible because the induced base-conversions occur at a much higher frequency and are therefore much more prevalent than background sources of mismatches to the reference sequence. Typical software capable of spotting induced base-conversions are using Samtools and htslib (https://github.com/samtools), Pysam (Python package; https://github.com/pysam-developers/pysam), Rsamtools (R Package; https://kasperdanielhansen.github.io/genbioconductor/html/Rsamtools.html) to efficiently load SAM/BAM files to compare read to reference sequence to identify read-level mismatches.

In some embodiments of the methods for determining the number of copies of one or more RNA molecule disclosed herein, Step (vi) comprises identifying, from the information in step (v), the number of unique molecule-specific base-conversion patterns that correspond to an RNA molecule with a particular sequence in the population of RNA molecules.

The first step of the process of determining the number of unique molecule-specific base-conversion patterns is pattern imputation. Each sequenced fragment is aligned to a subset of the sequence of an RNA molecule in the population of RNA molecules. As such, each molecule-specific base-conversion pattern is incomplete on a per-read basis. Accordingly, the full base-conversion pattern has to be imputed for each read. For example, reads can be aggregated to construct a matrix of conditional probabilities where each entry is the estimated probability of observing a base-conversion in that position given the known presence of a base-conversion in another position. The estimated probability is based on a Bayes estimator using the beta distribution as a conjugate prior distribution with parameters α=0.1, β=1 for the binomial distribution, p\hat{ }=(x+α)/(n+α+β), where x is the number of observed base-conversions conditioned on the n observed reads with a base-conversion in the other position. In general, a and B can be other values, as long as a is small and B is large. Such an estimator is used in order to account for positions which have no overlap in any reads, and this results in a small but non-zero probability of observing a base-conversion.

After this matrix is constructed, all observed base-conversions in each read are used to impute all positions using this conditional probability matrix. It should be noted that even positions which are observed in the read can be imputed to account for noise in the sequencing read out. There are two interesting imputations to make. The first one is the most likely value: if the presence of a base-conversion is the most likely or the absence of a base-conversion is the most likely. The second imputation is the imputed probabilities of observing a base-conversion in that position, which may be used to propagate the uncertainties in downstream analysis. The resulting imputed patterns can then be clustered using a preferred clustering algorithm.

Next, clustering of imputed patterns is performed. The clustering step serves two purposes: (i) counting the number of patterns present effectively counting the number of observed molecules, and (ii) grouping reads by molecule to be used for full-length reconstruction.

There are multiple options for clustering the imputed patterns, including Bernoulli mixture model and density-based clustering.

Bernoulli mixture model clustering treats each read as a composite of one or more binary patterns which are found through Expectation-Maximisation. Density-based clustering identifies the high-density areas of binary patterns and then connects points in this space by a distance metric. In the context of the methods disclosed herein, a distance metric for binary data is appropriate. For example, Dice dissimilarity, Hamming distance, Jaccard-Needham dissimilarity, Kulsinski dissimilarity, Rogers-Tanimoto dissimilarity, Russell-Rao dissimilarity, Sokal-Michener dissimilarity, Sokal-Sneath dissimilarity or Yule dissimilarity. Examples of algorithms in this category is DBSCAN and OPTICS. Another option is to cluster the imputed probabilities instead of the imputed patterns. The main consideration for the algorithms used in density-based clustering is how far away a point can be from a high-density area to be a part of that cluster. For instance, if a point is too far away from any high-density area it is not considered a part of any cluster. DBSCAN allows for a tuneable E parameter which regulates this, while OPTICS abstracts this parameter away and instead lets you set a minimum number of points which forms a cluster.

Determining the number of unique molecule-specific base-conversion patterns can be achieved by applying statistical model to all the molecule-specific base-conversion patterns of sequenced DNA molecules/fragments that align with the sequence of the RNA molecule of interest. The statistical model may be in the form of python programming language derived from packages such as SciPy (website: www.scipy.org). The key processing steps that such software must perform are: (i) retrieve base-conversion patterns for each DNA molecule/fragment; and, (ii) group fragments by base-conversion patterns by statistical methods. Examples of such statistical methods include but are not limited to: multivariate Bernoulli mixture model, density-based clustering, naive bayes, and random graph-based methods.

Another strategy to group sequences by their molecule-specific base-conversions patterns is to compare each sequence with a set of other sequences using a similarity measure. In the context of the present application, conversion patterns obtained per sequence or derived from one or more sequences are compared. For instance, mutual information or rand score metric may be used as a similarity metric. To avoid false positives, the similarity metric can be adjusted according to the actual number of overlapping eligible position found in the sequences and using a background model of similarity values that can arise due to chance alone. As an example, two conversion patterns from two reads which have many eligible positions that overlap are easy to statistically assign as arising from the same or different original molecules. However, two conversion patterns from two reads which only have three eligible positions that overlap may have a perfect match due to chance alone, and this must be controlled for. One such background model is the hypergeometric distribution model, which considers the number of overlapping eligible bases and the number of converted positions for both patterns in that overlapping region. Direct examples of adjusted similarity metrics are the adjusted mutual information and the adjusted rand score, where values close to 0 are consistent with the unadjusted similarity score occurring due to chance, and values close to 1 indicate a similarity not occurring due to chance. Using those adjusted similarity metrics, it is possible to accurately assign sequences according to their base conversion patterns over the full range of overlapping sequence lengths. More specifically, sequenced fragments are often ordered based on their genomic location and their base-conversion patterns. Each fragment can then be compared to all base-conversion patterns obtained from groups of previously analysed sequence fragments (or merges from previous such comparisons). Often, the threshold used for the adjusted similarity metric is in the range of 0.15-0.50. Higher values in that range result in stricter assignment of sequences to each other, whereas lower thresholds can give rise to larger number of false positives. Sufficiently good matches are often in the value range of 0.20-0.30, and higher values indicate an even better match. The presence of good, adjusted similarity values (i.e. above the set threshold) results in addition of the specific fragment to the one or more previously grouped sequences, and the addition of the specific base-conversion pattern in that sequence being added to that group. If there is no sufficiently good match (i.e. all comparisons yield a value below the set threshold), the fragment becomes a new group representing a unique molecule-specific base-conversion pattern. The use of such an approach simultaneously provides the number of unique molecule-specific base-conversion patterns, the actual patterns themselves, and the sequencing reads which constitute each pattern.

Using the molecule-specific base-conversion patterns produced by the methods disclosed herein, it is possible to either count RNA molecules after a successful (or partial) RNA sequence reconstruction or it is possible to skip RNA sequence reconstruction (e.g. if sequencing at lower sequence depths) and locally count RNA molecules based on the molecule-specific base-conversion patterns observed around a specific base pair of the DNA/RNA sequence. For example, all reads which cover a specific exon-exon junction of a gene may be collected. Then, the strategies for grouping read sequences by their molecule-specific base-conversion patterns which are described in the preceding paragraphs may be used to locally reconstruct molecules which span a specific exon-exon junction. Other features of interest may be the transcription start site or poly-adenylation site. Although counts obtained used the latter strategy may be an underestimate due to the limited sequencing depth, that approach could be valuable for applications such as diagnostics.

In some embodiments of the methods disclosed herein, one or more of Steps (i) to (iii) is performed in a droplet-based environment, a plate-based environment, attached to beads, or in-situ.

In some embodiments of the methods disclosed herein, the population of RNA molecules comprises one or more sequence variant of the same gene; or one or more allelic variant of the same gene; or one or more splice variant of the same gene; one or more RNA isoforms resulting from alternative use of promoters; or one or more RNA isoforms resulting from alternative use of splice sites; or one or more RNA isoforms resulting from alternative use of polyadenylation sites.

In a fifth aspect, the invention provides for the use of error-prone reverse transcription to generate, from a population of RNA molecules, a population of DNA molecules in which each DNA molecule comprises one or more base-conversion relative to the corresponding RNA molecule and has a molecule-specific base-conversion pattern, for determining the number of copies of one or more RNA molecule in a population.

The first and second aspects disclosed herein provide examples of methods in which error-prone transcription is used to generate a population of DNA molecules for determining number of copies of one or more RNA molecule in a population.

In a sixth aspect, the invention provides for the use of error-prone reverse transcription to generate, from a population of RNA molecules, a population of DNA molecules in which each DNA molecule comprises one or more base-conversion relative to the corresponding RNA molecule and has a molecule-specific base-conversion pattern, for determining the sequence of one or more RNA molecule in a population.

The third and fourth aspects disclosed herein provide examples of methods in which error-prone transcription is used to generate a population of DNA molecules for determining number of copies of one or more RNA molecule in a population.

In a seventh aspect, the invention provides a population of DNA molecules obtained or obtainable by a method of the first, second, third, or fourth aspect, or by the use of the fifth or sixth aspects.

In an eighth aspect, the invention provides a kit for performing error-prone reverse transcription, wherein the kit comprises:
(i) a reverse transcriptase enzyme;
(ii) one or more base analogue; and,
(iii) instructions for use.

In some embodiments of the kits disclosed herein, the one or more base analogue is selected from the group consisting of: 2′-deoxy-P-nucleoside-5′-triphosphate (dPTP); 8-Oxo-2′-deoxyguanosine-5′-triphosphate (8-oxo-GTP); 2-Thiothymidine-5′-triphosphate (2-thioTTP), 5-Formyl-2′-deoxyuridine-5′-triphosphate, 5-Propynyl-2′-deoxycytidine-5′-triphosphate, 5-Iodo-2′-deoxycytidine-5′-triphosphate, 5-Propargylamino-2′-deoxyuridine-5′-triphosphate, or combinations thereof.

In some embodiments of the kits disclosed herein, the reverse transcriptase is an error-prone reverse transcriptase.

In some embodiments of the kits disclosed herein, the kit further comprises a composition comprising dNTPs.

In some embodiments of the kits disclosed herein, the kit further comprises an oligonucleotide primer composition suitable for use in reverse transcription. In some embodiments, the oligonucleotide primer composition comprises oligo-dT primers, random hexamer primers, or gene-specific primers.

In some embodiments of the kits disclosed herein, the kit further comprises compounds that can modify bases on the first strand cDNA. In some embodiments, the compounds deaminate nitrogenous bases, for example using bisulfite.

In a ninth aspect, the invention provides a method, or a use, or a population of DNA molecules, or a kit substantially as described herein with reference to the accompanying description, examples, claims and figures

### DESCRIPTION OF THE FIGURES

Embodiments of the present invention will now be described, by way of example only, with reference to the accompanying figures, in which:

FIG. 1 shows the core technologies that can be used to obtain cDNA with molecule-identifying conversion patterns. (A) Direct and erroneous incorporation of a canonical base in the first-strand cDNA molecule, for example by an error-prone reverse transcriptase. (B) The incorporation of a promiscuous base analogue in the first-strand cDNA during reverse transcription. During second-strand synthesis, an erroneous canonical base can be incorporated thus giving rise to an error on that position. (C) The incorporation of protective or chemical-sensitive base analogue in the first-strand cDNA during reverse transcription. Subsequent chemical or enzymatic treatment either modifies the base analogue or the corresponding canonical base. During second strand synthesis, this can give rise to the incorporation of an erroneous canonical base, which can be detected as an error on this position.

FIG. 2 shows the core steps of the methods of the present invention and explains how base-conversion patterns can be used to identify sequences from the same initial RNA molecule. By introducing random base-conversions during the synthesis of cDNA from RNA molecules, the RNA molecules can be counted, and the RNA sequence can be reconstructed.

FIG. 3 (A-C) Genome browser screenshots of single-cell RNA-sequencing data for a representative cell (generated according to Smart-seq3 technology) with induced base-conversions for genes MED27, GUK1 and AP2M1 respectively. In the corresponding experiment, 0.5 mM 2′-deoxy-P-nucleoside-5′-triphosphate (dPTP) was used to induce base-conversions and the induced base-conversion patterns uniquely mark each read originating from the same initial RNA molecule, with reads being grouped to particular molecules based on their 5′ molecular barcodes.

FIG. 4 shows that reverse transcription in the presence of the base analogue dPTP can give rise to useful levels of base-conversions and that the stability of those base-conversions in subsequent steps depends on efficient removal of the base analogue after the reverse transcription step. It will be understood that the conversion identity is written with the original reference base in lower-case, and the new base as upper-case. For example, a G-to-A conversion can be written as gA. (A) Reverse transcription in the presence of the base analogue dPTP gives rise to high levels of base-conversions as long as the base analogue (dPTP) is efficiently removed after reverse transcription either by bead clean-up or by treatment with alkaline phosphatase (FastAP). This panel also shows that the type of base-conversion event is dependent on the strand the gene is encoded. (B) Illustration that the stability of the base-conversions in sequencing reads corresponding to the same molecule depends on the efficient removal of base analogue after reverse transcription by either bead clean-up or by treatment with alkaline phosphatase (FastAP).

FIG. 5 shows simulation results for the number of unique base-conversions patterns expected (y-axis) in experiments with different base-conversion fractions (x-axis) and different overlaps in DNA fragments (50-200 bp; the individual curves within each figure). The expected number of base-conversion patterns was computed for a gene expressed at different RNA copy numbers (10, 100 or 1000; columns) and for experiments where one to four of the bases present in a molecule could have been converted (1st row: one base; 2nd row: two bases, such as the case for dPTP; 3rd row: three bases; 4th row: all four bases) with the same specified individual base-conversion fraction (as shown on the x-axis) applied to 1, 2, 3, or 4 bases (as indicated in the rows). The dashed lines show the base-conversion fraction of 0.04.

FIG. 6 shows that the amounts of dPTP-induced base-conversions on the positive strand positively correlate with the applied dose of dPTP during reverse transcription. It will be understood that the conversion identity is written with the original reference base in lower-case, and the new base as upper-case. For example, a G-to-A conversion can be written as gA.

FIG. 7 shows that the base analogue dPTP can be incorporated into cDNA on RNA attached to beads that was captured in droplets using MGI C4. Reverse transcription was performed with added dPTP, and PCR amplification was carried out using KAPA HiFi PCR enzyme. It will be understood that the conversion identity is written with the original reference base in lower-case, and the new base as upper-case. For example, a G-to-A conversion can be written as gA. Note that in this figure, and the figures below, unless stated otherwise, base conversion rates that are shown are for features on the positive strand.

FIG. 8 shows base-conversions that are induced by the incorporation of different base analogues during reverse transcription. It will be understood that the conversion identity is written with the original reference base in lower-case, and the new base as upper-case. For example, a G-to-A conversion can be written as gA. (A) Base-conversions obtained by the incorporation of 2-thioTTP during reverse transcription (performed in biological duplicates). The experimental details for the data shown in this figure are described in Example 5 below. (B) Base-conversions obtained by the incorporation of 5-Formyl-2′-deoxyuridine-5′-triphosphate, 5-Propynyl-2′-deoxycytidine-5′-triphosphate, 5-Iodo-2′-deoxycytidine-5′-triphosphate, or 5-Propargylamino-2′-deoxyuridine-5′-triphosphate during reverse transcription.

FIG. 9 shows all induced base conversions for different second-strand synthesis approaches that were performed on cDNA containing dPTP, 5-Formyl-dUTP, or canonical bases only (H₂O results). It will be understood that the conversion identity is written with the original reference base in lower-case, and the new base as upper-case. For example, a G-to-A conversion can be written as gA.

FIG. 10 shows that different PCR enzymes efficiently incorporate canonical dNTPs opposite non-canonical bases in cDNA. It will be understood that the conversion identity is written with the original reference base in lower-case, and the new base as upper-case. For example, a G-to-A conversion can be written as gA.

FIG. 11 shows that the incorporation of non-canonical bases during reverse transcription (here using a methylated cytosine base), combined with bisulfite treatment of cDNA (which results in the conversion of unmethylated cytosines to uracil), can give rise to base-conversions in a highly controlled manner. It will be understood that the conversion identity is written with the original reference base in lower-case, and the new base as upper-case. For example, a G-to-A conversion can be written as gA.

FIG. 12 shows RNA reconstructions results in the context of single-cell RNA-sequencing (see Example 8). (A) Histogram and density plot of the fraction of internal reads that could be assigned to 5′ anchored read pair based on the dPTP induced base-conversions. An internal read is classified as a paired-end sequenced read with the first reads not originating from the RNA 5′ end, so that both read fragments captured internal parts of the RNA. (B) Line plot with the lengths of reconstructed RNAs in experiment 5 (with and without assigning the internal reads to 5′ anchored reads based on induced base-conversion patterns) compared against long-read sequencing of similar cDNA libraries (sequencing here by Pacific Biosystems Sequel instrument). Reconstruction based on dPTP-induced base-conversions enabled internal reads to be assigned to 5′ anchored RNA reads to reconstruct approximately 1,250 bp of cDNAs at similar qualities to long-read sequencing technologies.

FIG. 13 shows that dPTP induced base-conversion in single-cell RNA-sequencing data can be used to assign sequenced reads to the correct strand. (A) Observed base-conversions when separating genes according to their location on the positive or negative strand of a DNA molecule. Two conversions (A-to-G and G-to-A) were specifically induced in genes located on the positive strand (and the reverse complement conversions for genes located on the negative strand). (B) The log-likelihood ratio of each partially reconstructed sequence to be assigned to the correct strand based on the base-conversions induced by 0.5 mM dPTP. The log-likelihood distributions for reads assigned to genes on positive or negative strand separate, demonstrating that the induced base-conversions contain the information needed to correctly assign the majority of reads to the correct strand.

FIG. 14 provides a schematic representation of an application in which the method of the present invention is used to count and reconstruct RNA sequences from single cells, in the context of Smart-seq3.

FIG. 15 provides a schematic representation of an application in which the method of the present invention is used to count and reconstruct RNA sequences from single cells, in the context of a novel early pooling based full-length transcriptome sequencing method. In such an application, the method of the present invention can both enable RNA counting and sequence reconstruction in a highly parallel manner to characterise large numbers of single cells.

FIG. 16 illustrates the cell-barcoding approach used in Example 10. In that approach, not all of the obtained reads contain cell-barcode (and UMI) information and so such experiments depend on molecular pattern identification in order to link reads to their corresponding cell barcodes.

FIG. 17 shows dPTP-mediated conversions obtained in a single cell experiment using an early pooling as illustrated in FIG. 16 (see Example 10). It will be understood that the conversion identity is written with the original reference base in lower-case, and the new base as upper-case. For example, a G-to-A conversion can be written as gA.

FIG. 18 shows the cumulative distribution of reconstructed molecule lengths across the whole experiment (n=96 cells) described in Example 10.

FIG. 19 shows the fraction of reads without a cell barcode which are successfully linked to a reconstructed molecule, for each gene with more than 50 molecules detected over all cells (n=6,684 genes). Centre lines denote the median; hinges denote the first and third quartiles; whiskers denote 1.5× the interquartile range (IQR).

FIG. 20 shows a representative screenshot from the Integrated Genome Viewer (genome browser) of individual reads as well as a reconstructed molecule from the mouse gene Psma2 in a single cell, using mismatches induced by 4-thio-uridine labelling during cell culturing.

FIG. 21 shows that adding dATP during second-strand synthesis creates a sub-optimal and unbalanced mix of dNTP concentrations and thereby results in the favouring of one conversion type over another (i.e. G-to-A conversions over A-to-G conversions). (A) Rates of G-to-A conversions observed in “no added dATP” and “Added dATP” replicates. (B) Rates of A-to-G conversions observed in “no added dATP” and “Added dATP” replicates. It will be understood that the conversion identity is written with the original reference base in lower-case, and the new base as upper-case. For example, a G-to-A conversion can be written as gA.

### EXAMPLES

### Example 1

### Materials and Methods

Single human K562 cells were sorted into individual wells of a 384-well plate containing 3 UL Vapor-Lock (Qiagen) and 0.3 μL Smart-seq3 lysis buffer (see: Hagemann-Jensen et al, 2020. Nature Biotechnology, 38:708-714) with either 0 or 0.5 mM dPTP added. Reverse transcription was performed as described in Hagemann-Jensen et al, 2020 (i.e. Smart-seq3 approach) with the exception of a 10-fold reduction in volumes, the reduction of dNTP concentrations to 0.1 mM each, and the MgCl₂ concentration being adjusted to 1.5 mM. The final volume in the reverse transcription was 0.4 μL. For the Dilution condition, 4.6 μL of nuclease-free water was added to the reaction. For the wells treated with alkaline phosphatase, 0.1 μL of FastAP (Thermo Scientific 0.2 U/μL) was added to the reaction which was then incubated at 37° C. for 20 minutes and 75° C. for 10 minutes to inactivate the FastAP enzyme. For the bead purification, the volume was adjusted to 10 μL and clean-up was performed with 8 μL SPRI paramagnetic beads.

The purified cDNA was eluted in 5 μL. A PCR mastermix was then added to a final volume of 5 μL, 0.5 μL, 5 μL, and 0.5 μL for the bead clean-up, FastAP, Dilution, and no clean-up conditions respectively. PCR was performed as described in Hagemann-Jensen et al, 2020 with the exception of the presence of varying amounts of salts and enzymes carried over from the reverse transcription and FastAP reactions for the different conditions. The libraries obtained were tagmented using Illumina Nextera XT chemistry and amplified. The resulting library was circularised using the MGI App-A conversion kit and then sequenced on the MGI DNBSEQ-G400 platform using a StandardMPS PE100 kit.

Data was processed using zUMIs (Parekh et al, 2018. Gigascience, 2018 Jun. 1; 7 (6): giy059. doi: 10.1093/gigascience/giy059). The option find_pattern ATTGCGCAATG (SEQ ID NO: 5) was specified to identify UMI-containing 5′-reads and all reads were mapped using STAR to perform alignment with the human genome (hg38). STAR settings were changed to allow for up to 20% mismatches. Note that mismatches here correspond to all possible mismatches.

### Results

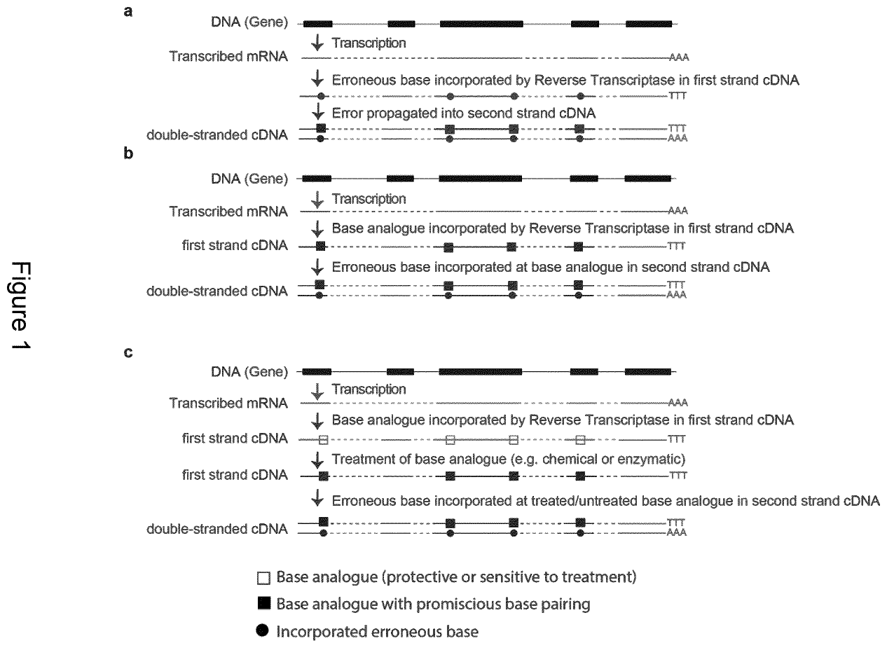

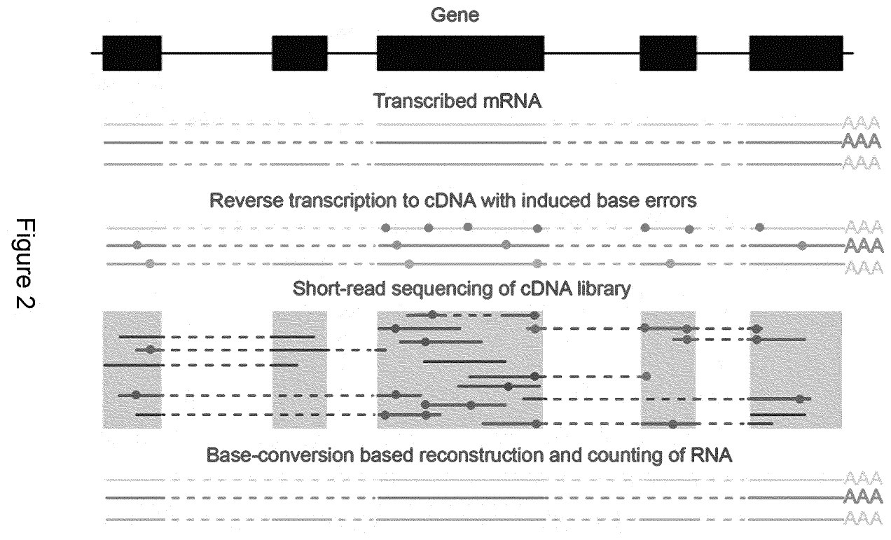

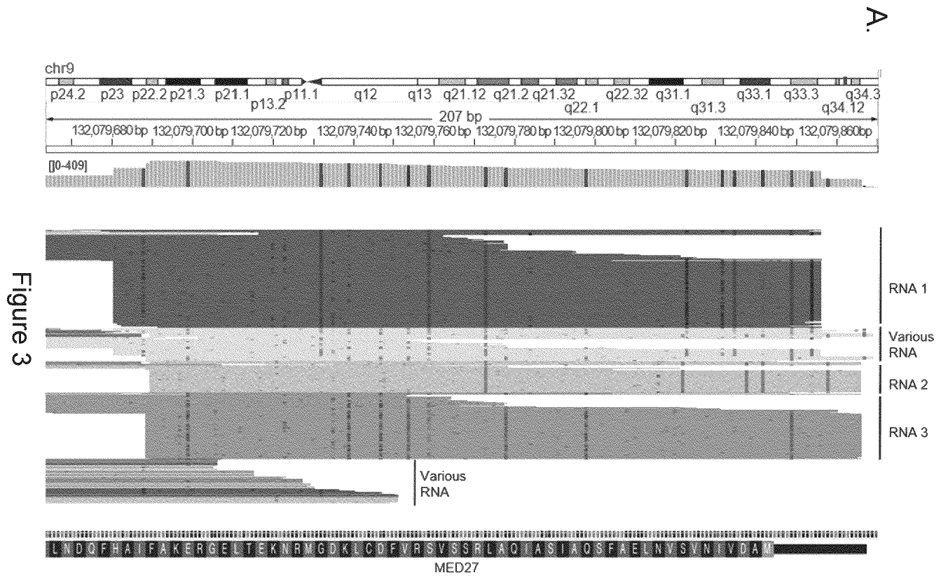

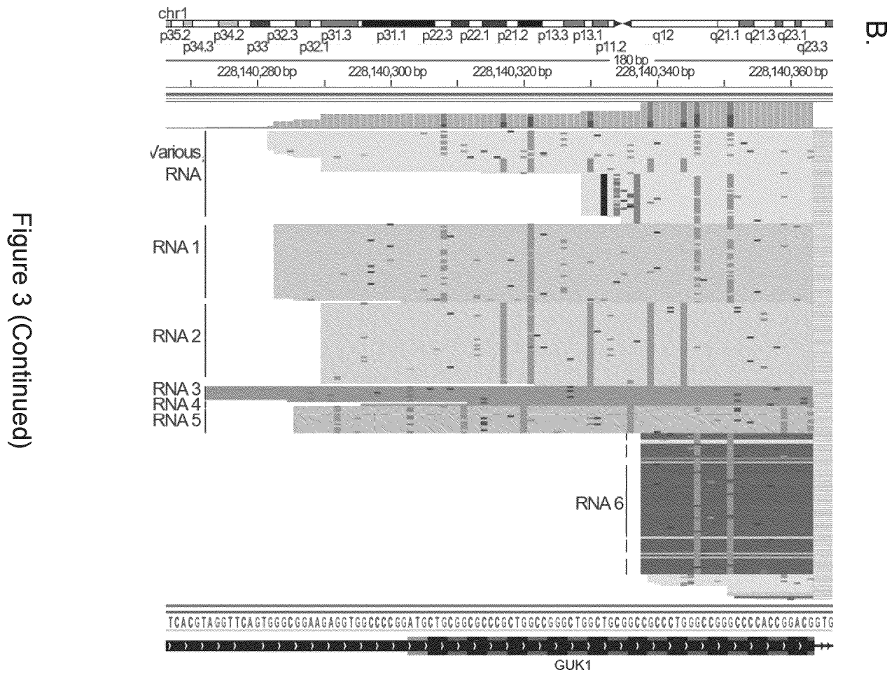

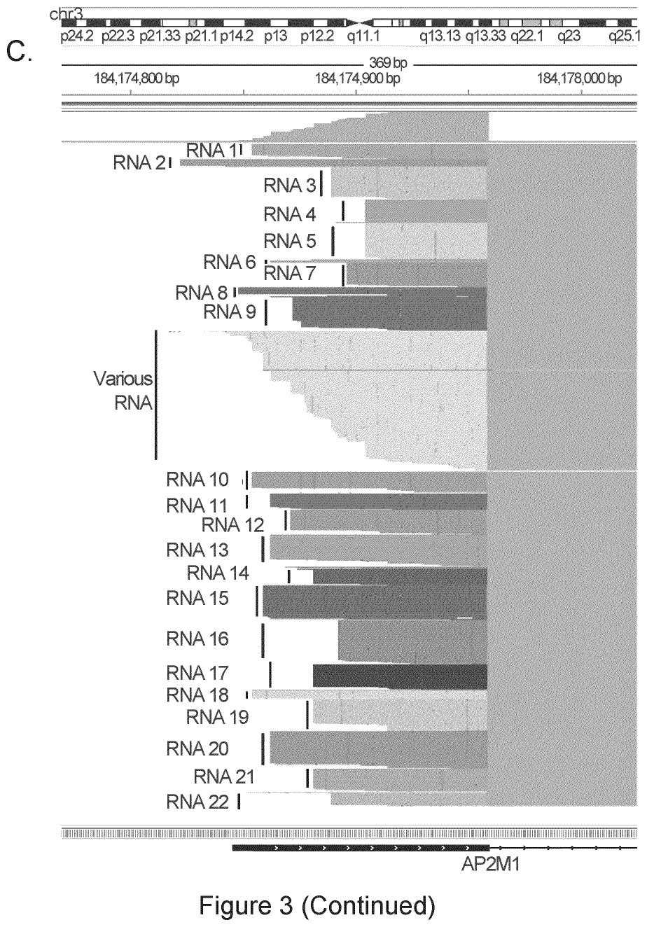

The use of error-prone reverse transcription, through the strategies described in the present application, gives rise to the presence of unique patterns in the cDNA molecules produced (FIG. 1). Those patterns can then be used for the identification of the molecule of origin in downstream applications such as molecule counting or molecule reconstruction (FIG. 2). In Example 1 we show that these patterns can be created through the use of dPTP during reverse transcription reactions (FIG. 3). Furthermore, this example confirms that those patterns uniquely identify the molecule of origin since the patterns corresponds well with the unique molecular identifier (UMI) that was used in this experiment.

Remaining and free dPTP needs to be removed before PCR can be performed to avoid the incorporation of base analogues during cDNA amplification. This important since the incorporation of base analogues during PCR leads to production of base-conversion patterns that do not uniquely correspond to an individual RNA molecule and would therefore not be useful in downstream analysis.

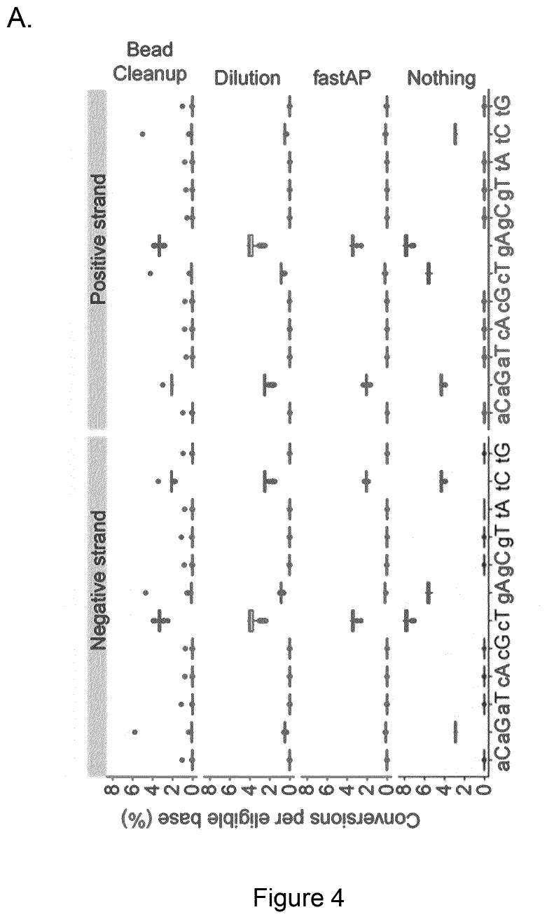

Importantly, the conversions induced by dPTP can be readily detected since the incorporation of dPTP during reverse transcription can only give rise to aG and gA conversions for features that are located on the positive strand, and cT and tC conversions for features that are located on the negative strand. In the absence of any clean-up strategy, both pairs of possible conversions are seen for both positive strand and negative strand features (FIG. 4A), indicating that the dPTP was incorporated during PCR instead of reverse transcription. However, cleaning up with SPRI paramagnetic beads as well as treatment with alkaline phosphatase (fastAP) reduces the conversion rates that correspond to incorporations that occurred in the amplification of cDNA (i.e. tC and cT conversions in features located on the positive strand and aG and gA conversions in features located on the negative strand). In addition, the base-conversion patterns in samples where free dPTP was efficiently removed (either by fastAP or SPRI paramagnetic bead clean-up) are stable, as opposed to those samples where no clean-up was performed (FIG. 4B).

### Example 2

### Materials and Methods

Single human K562 cells were sorted into individual wells of a 384-well plate containing 0.3 μL Smart-seq3 lysis buffer with dNTPs present at 0.1 mM and added varying concentrations of dPTP. The concentrations of dPTP that were present during the respective reverse transcription reactions were 0 mM, 0.25 mM, 0.5 mM, 1 mM. After reverse transcription (as in Example 1), FastAP (Thermo Scientific) was added to a final concentration of 0.1 U/μL in a total volume of 0.5 μL. The reactions were incubated at 37° C. for 20 minutes and FastAP was inactivated at 72° C. for 10 minutes. PCR, tagmentation, and subsequent amplification was performed as described in Example 1 above. The resulting library was circularised using the MGI App-A conversion kit and sequenced on the MGI DNBSEQ-G400 platform using a StandardMPS SE100 kit.

Data was processed using zUMIs (Parekh et al, 2018. Gigascience, 2018 Jun. 1; 7 (6): giy059. doi: 10.1093/gigascience/giy059). The option find_pattern ATTGCGCAATG (SEQ ID NO: 5) was specified to identify UMI-containing 5′-reads and all reads were mapped using STAR to perform alignment with the human genome (hg38). STAR settings were changed to allow for up to 20% mismatches. Note that mismatches here correspond to all possible mismatches.

### Results

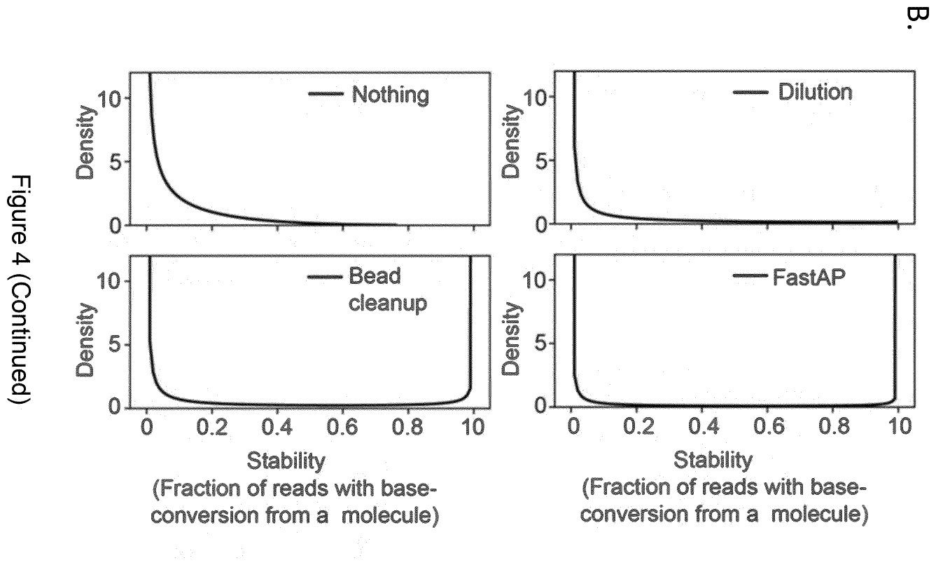

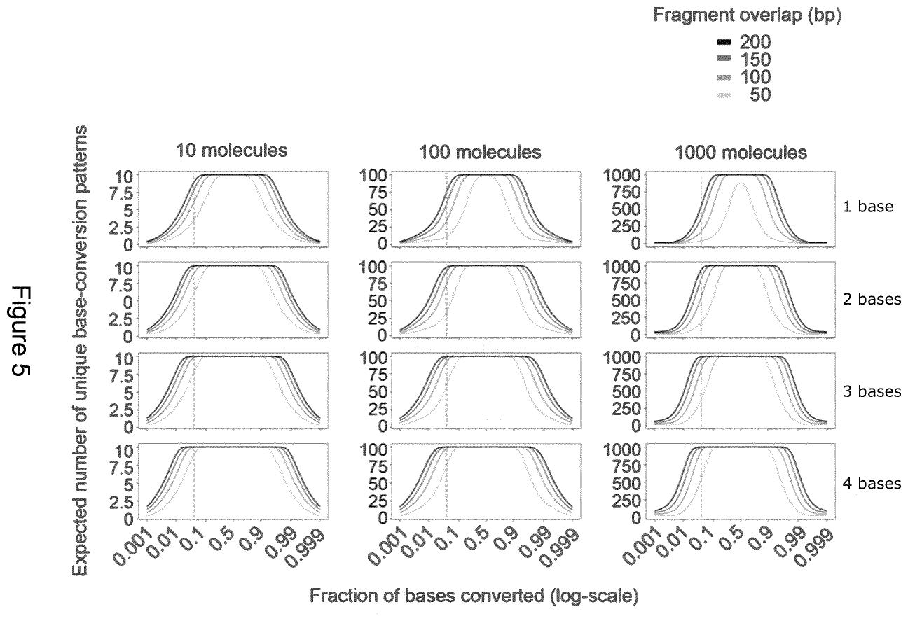

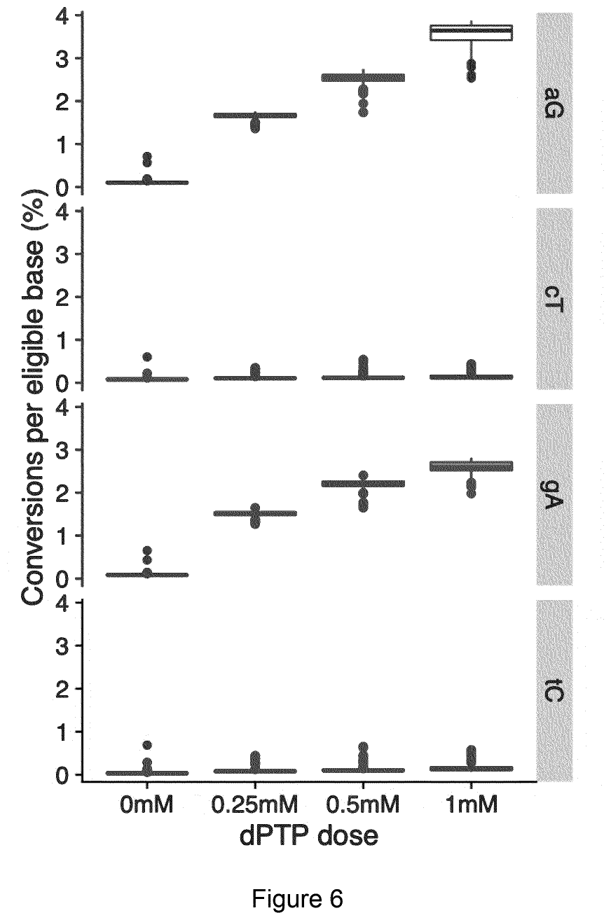

The efficiency of the introduction of errors into cDNA is important for producing sufficient unique patterns to allow identification of molecules derived from a particular original RNA molecule in the methods of the present invention (FIG. 5). This example shows that reaction conditions, and specifically the concentration of the base analogue dPTP, can be tuned to obtain high percentages of base-conversion events (FIG. 6). Furthermore, this example shows that efficient reverse transcription of RNA from single cells can be performed in the presence of those concentrations of dPTP.

### Example 3

### Materials and Methods

120,000 K562 cells were encapsulated and lysed in droplets as per the standard protocol of the MGI C4 DNBelab. RNA capture and cleaning was performed as per the standard protocol. The reaction was then split in two and reverse transcription was performed according to the standard Smart-seq3 protocol (Hagemann-Jensen et al, 2020) in 50 μL reactions with the concentration of each dNTP at 0.1 mM and the use of the RT primer mix from the MGI C4 DNBelab kit. For one of the two samples, 1 mM dPTP was added. Reverse transcription was performed according to the standard protocol. The resulting reaction was then cleaned up according to the standard MGI C4 DNBelab protocol. PCR amplification was performed using KAPA HiFi in the presence of 10 mM of each dNTP and a total of 4 μL of MGI C4 DNBelab cDNA amplification primer mix per sample. 200 ng of the resulting cDNA library was tagmented using Illumina Nextera XT at ⅕ volume. 200 pg of the resulting cDNA library may also be used. The resulting library was circularised using the MGI App-A conversion kit and sequenced on the MGI DNBSEQ-G400 platform using a SE100 kit.

Data was processed using zUMIs (https://github.com/sdparekh/zUMIs), using STAR to perform alignment with the human genome (hg38). STAR settings were changed to allow for up to 20% mismatches. Note that mismatches here correspond to all possible mismatches.

### Results

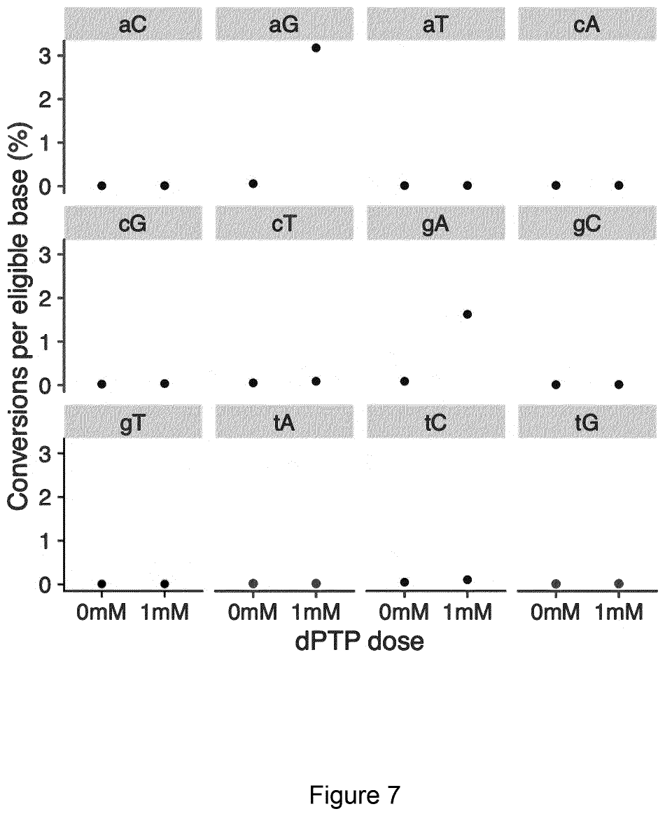

Single-cell transcriptomics methods are broadly separated into plate-based methods and droplet-based methods. While plate-based methods rely on the separation of cells into separate well of multiwell plates, droplet-based methods instead utilise lipid-droplets in which cells are physically separated from each other. This example shows that performing error-prone reverse transcription by incorporation of dPTP in a droplet-based single-cell library preparation protocol (C4 DNBelab, MGI technologies) can result in high percentages of base conversions (FIG. 7). This demonstrates that in addition to plate-based methods (as shown in the examples above), droplet-based methods are also compatible with error-prone reverse transcription as described in the present application.

### Example 4

### Materials and Methods

Purified, DNAse-treated RNA was reverse transcribed in the presence of 2-Thio-dTTP (TriLink Biotechnologies N-2035) at 2 mM using modified Smart-seq3 reaction conditions (as in Example 1). Alkaline phosphatase treatment of the reaction was performed using FastAP (Thermo Scientific) at a final concentration of 0.04 U/μL. The reaction was incubated at 37° C. for 20 minutes and FastAP was then inactivated at 75° C. for 10 minutes. PCR, tagmentation, and indexing PCR were then performed as described in Example 1 above. The resulting library was sequenced on the Illumina NextSeq500 platform using a 75-cycle High Output kit v2.5.

Data was processed using zUMIs (https://github.com/sdparekh/zUMIs), using STAR to perform alignment with the human genome (hg38). STAR settings were changed to allow for up to 20% mismatches. Note that mismatches here correspond to all possible mismatches.

### Results

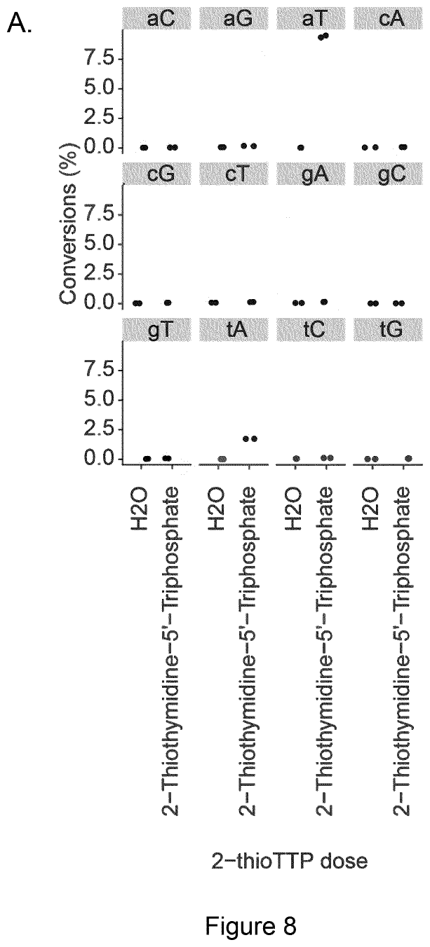

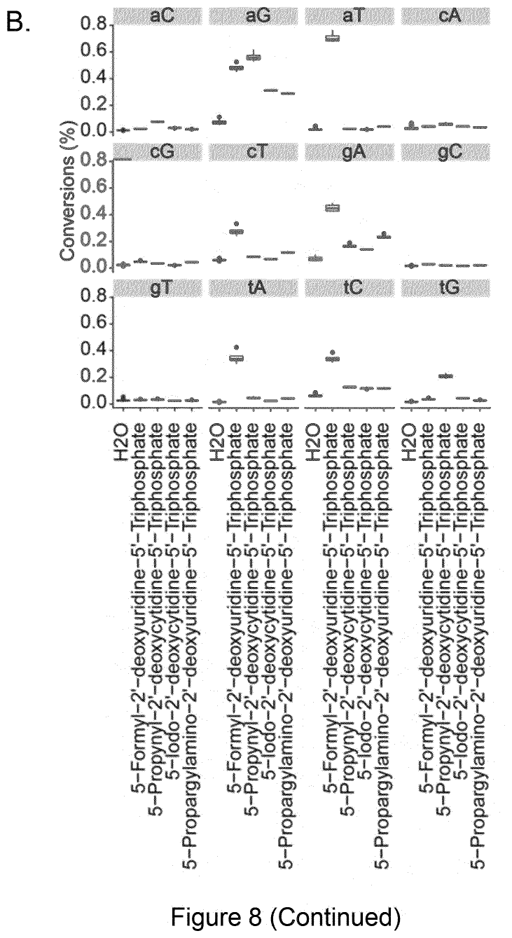

This example demonstrates that the incorporation of 2-thio-dTTP during reverse transcription can give rise to high percentages of base-conversion events (FIG. 8A).

### Example 5

### Materials and Methods

4 ng of purified DNAse-treated RNA was reverse transcribed using Maxima H-minus reverse transcriptase (5% Poly-Ethylene Glycol 8000, 0.1% Triton X-100, 5 U/μL Recombinant RNAse Inhibitor, 0.1 mM dNTPs each, 25 mM Tris-HCL, 30 mM NaCl, 1.5 mM MgCl, 1 mM GTP, 8 mM DTT, Smart-seq2 oligo-dT 0.5 μM, Smart-seq2 template switch oligo 2 μM (see: Picelli et al, 2013. Nature Methods, 10:1096-1098), Maxima H-minus Reverse Transcriptase 2 U/μL). The names and product numbers of the different analogues that were tested in this experiment were 5-Formyl-2′-deoxyuridine-5′-triphosphate (TriLink Biotechnologies N-2067), 5-Propynyl-2′-deoxycytidine-5′-triphosphate (TriLink Biotechnologies N-2016), 5-Iodo-2′-deoxycytidine-5′-triphosphate (TriLink Biotechnologies N-2023), and 5-Propargylamino-2′-deoxyuridine-5′-triphosphate (TriLink Biotechnologies N-2062). The base analogues were present in concentrations of either 4 mM or 0.25 mM during reverse transcription. Base analogues were dephosphorylated by treating with 0.12 U FastAP (Thermo Scientific) for 20 minutes at 37° C., followed by FastAP inactivation at 75° C. for 10 minutes. PCR was performed according to Smart-seq3 standard protocol (see: Hagemann-Jensen et al, 2020), with the exception of the use of ISPCR primer instead of the standard Smart-seq3 forward and reverse primers. The DNA libraries were tagmented and indexed as described in Example 1 above. The resulting library was circularised using the MGI App-A conversion kit and sequenced on an MGI DNBSEQ-G400 platform using a StandardMPS PE200 kit.

Data was processed using zUMIs (https://github.com/sdparekh/zUMIs), using STAR to perform alignment with the human genome (hg38). STAR settings were changed to allow for up to 20% mismatches. Note that mismatches here correspond to all possible mismatches.

### Results

This example shows that four additional base analogues can give rise to conversions with varying efficiencies (FIG. 8B). Although individually the error-rates that are obtained by these base analogues are relatively low, utilising a combination of base analogues can raise the effective overall conversion-rate.

### Example 6

### Materials and Methods

20 ng of DNAse-treated RNA was reverse transcribed according the Smart-seq2 reaction conditions (Picelli et al, 2013) with each dNTP concentrated at 0.1 mM and in the presence of dPTP (0.5 mM), the presence of 5-Formyl-dUTP (0.25 mM), or in the absence of any base analogue. The resulting cDNA was purified with AMPure SPRI paramagnetic beads (1:1 bead to cDNA volume ratio) and eluted in a final volume of 120 μL. For each condition, 2 μL of purified cDNA was used for second strand synthesis with Klenow, T4, or water as a negative control. In addition to the enzyme or water negative control, the reaction consisted of 1×NEB buffer 2, 0.2 mM of each dNTP, and 0.2 μM ISPCR primer. The reaction was incubated for 2 hours at 37 C. The second-strand product was then amplified using KAPA according to the Smart-seq2 protocol (Picelli et al, 2013) in the presence of 0.4 μM ISPCR primer and 1 mM of each dNTP in a total reaction volume of 10 μL for 24 cycles. The resulting libraries were tagmented according to the Smart-seq3 protocol (Hagemann-Jensen et al, 2020), circularised using the MGI App-A conversion kit, and sequenced on a MGI DNBSEQ-G400 platform using a StandardMPS SE100 kit.

Data was processed using zUMIs (https://github.com/sdparekh/zUMIs), using STAR to perform alignment with the human genome (hg38). STAR settings were changed from standard to allow for up to 20% mismatches. Note that mismatches here correspond to all possible mismatches.

### Results

The incorporation of base analogues in the first strand cDNA (FIG. 1) directly results in the incorporation of the incorrect canonical base during the synthesis of the second-strand CDNA. This example shows that the method chosen for second-strand cDNA synthesis affects the achieved conversion rate (FIG. 9). The addition of a separate second-strand synthesis step (before the cDNA amplification with KAPA) with Klenow DNA polymerase (but not with T4 DNA polymerase) increases the conversion rate for both dPTP and 5-Formyl-dUTP.

### Example 7

### Materials and Methods

100 ng of DNAse-treated RNA was reverse transcribed with Maxima H-minus reverse transcriptase (5% Poly-Ethylene Glycol 8000, 0.1% Triton X-100, 5 U/μL Recombinant RNAse Inhibitor, 0.1 mM dNTPs each, 25 mM Tris-HCL, 30 mM NaCl, 1.5 mM MgCl, 1 mM GTP, 8 mM DTT, Smart-seq2 oligo-dT 0.5 μM, Smart-seq2 template switch oligo 2 μM (see: Picelli et al, 2013. Nature Methods, 10:1096-1098), Maxima H-minus Reverse Transcriptase 2 U/μL) in the presence of 1 mM dPTP base analogue. The cDNA was purified using SPRI beads at a 0.8:1 ratio. The cDNA was then amplified using the following PCR enzymes; KAPA HiFi HotStart PCR enzymes (KAPA BioSystems KK2501), Phusion HF HotStart II (Thermo Scientific F459), NEBNext (NEB M0541), Q5 DNA polymerase (NEB M0491), Q5 Ultra II (NEB M0543), Platinum Superfi II (Thermo Scientific 12361010), Platinum II (Thermo Scientific 14966005), Terra Polymerase (Takara ST0287), VeriFi Polymerase (PB10.45), Amplitaq Gold (8080240), Taq DNA Polymerase (Invitrogen 18038-042). All PCRs were performed according to the manufacturer's protocols using the appropriate concentration of the ISPCR primer (Picelli et al, 2013). All DNA libraries were then purified using SPRI beads at 0.8:1 ratio. The resulting DNA was tagmented as described in Example 1 above. The resulting library was circularised using the MGI App-A conversion kit and sequenced on an MGI DNBSEQ-G400 platform using a StandardMPS SE100 kit.

Data was processed using zUMIs (https://github.com/sdparekh/zUMIs), using STAR to perform alignment with the human genome (hg38). STAR settings were changed to allow for up to 20% mismatches. Note that mismatches here correspond to all possible mismatches.

### Results

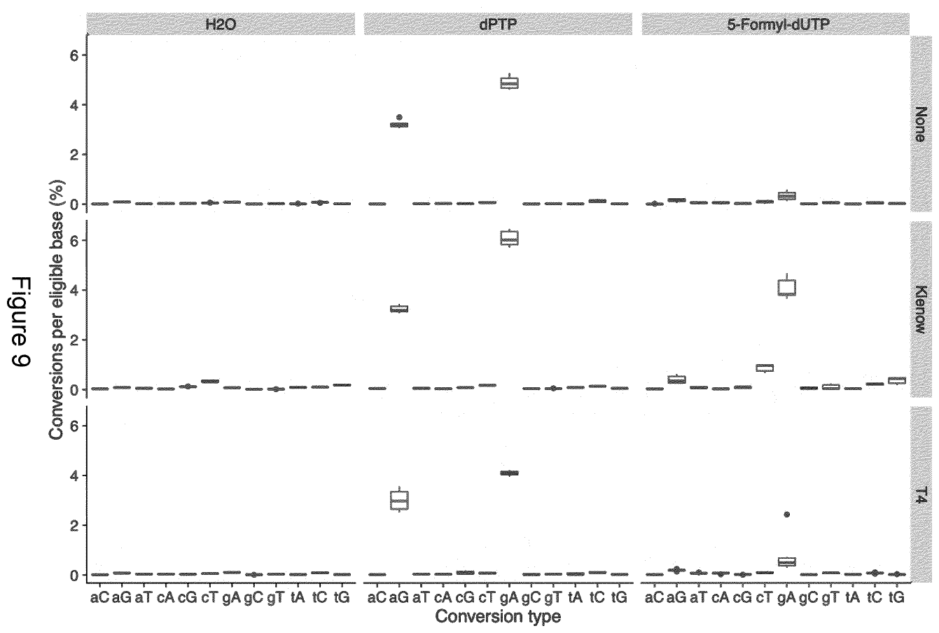

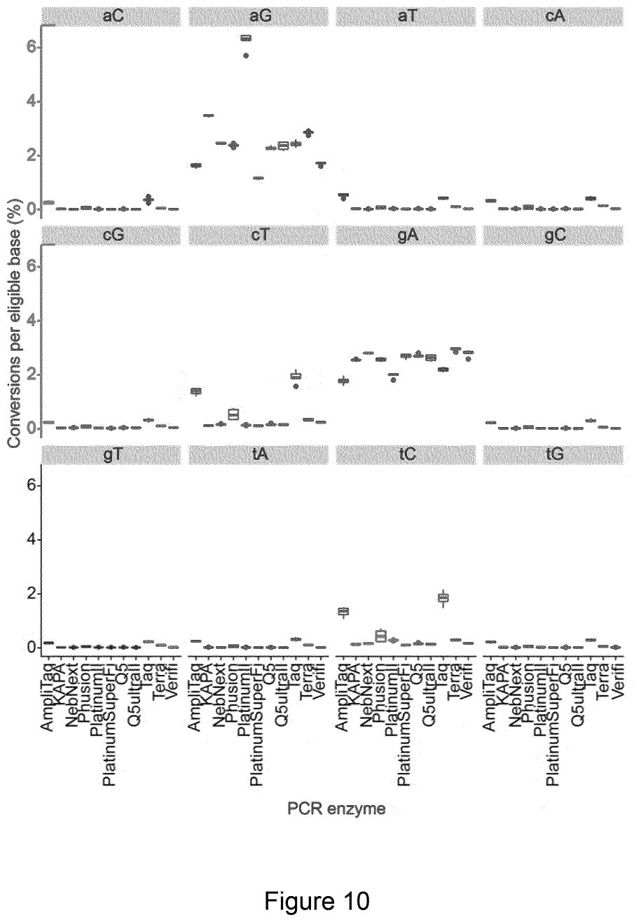

Most widely used single-cell RNA-seq library preparation strategies do not perform dedicated second-strand synthesis. The second strand is instead synthesised in the first cycle of the cDNA amplification PCR thereby effectively streamlining the protocol and increasing sensitivity. Considering the importance of second-strand synthesis (FIG. 10), the choice of the PCR enzyme is potentially of great importance. This example shows that the choice of PCR enzyme is indeed an important factor affecting the rate at which errors can be induced when amplifying cDNA containing dPTP. Furthermore, building on the results discussed in Example 6 above (and shown in FIG. 9), this example suggests that the cDNA amplification strategy can be tailored to the specific base analogue that is used.

### Example 8

### Materials and Methods

1.1 μg of purified and DNAse-treated RNA was reverse transcribed using Superscript II (Thermofisher) according to the manufacturers protocol in the presence of varying percentages of the CTP in the dNTP mix replaced by 5′-methyl-CTP. The percentages of 5′-methyl-CTP used were 0%, 20%, 50%, 80%, and 100% respectively. The resulting cDNA was bisulfite converted using the EZ DNA Methylation-Gold Kit (Zymo Research) according to the manufacturers protocol. Second strand synthesis was performed using Klenow (NEB) according to the manufacturers protocol with random hexamer primers. The second strand synthesis reaction was ended by adding EDTA to a final concentration of 10 mM and the resulting double-stranded DNA was purified using SPRI beads (1:1 ratio). The resulting DNA libraries were quantified and tagmentation was performed with Illumina Nextera XT using the manufacturers protocol but at ⅕ of the total volumes. The resulting libraries were circularised using the MGI App-A conversion kit and sequenced on an MGI DNBSEQ-G400 platform using a StandardMPS SE100 kit.

Data was processed using zUMIs (https://github.com/sdparekh/zUMIs), using STAR to perform alignment with the human genome (hg38). STAR settings were changed to allow for up to 40% mismatches. Note that mismatches here correspond to all possible mismatches.

### Results

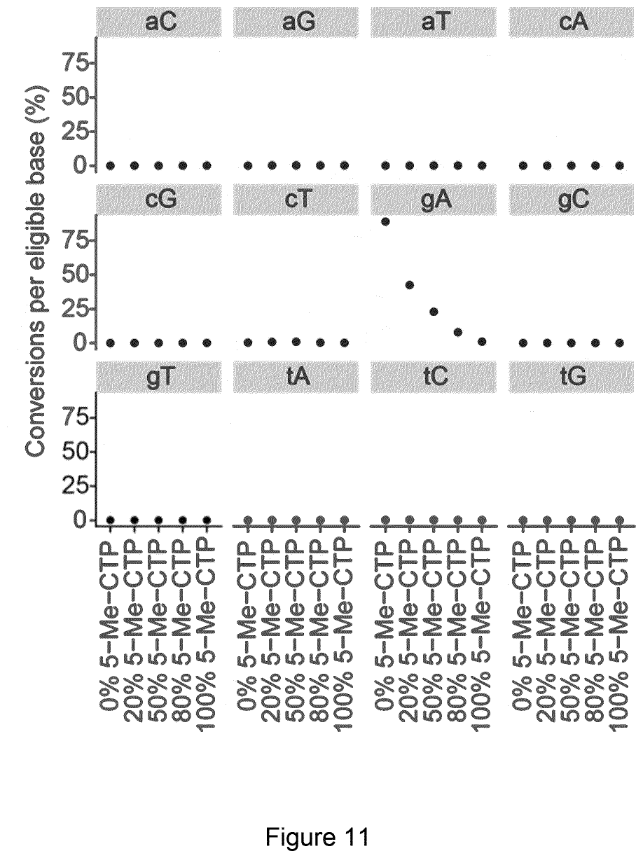

The bisulfite conversion of non-methylated cytosines in DNA results in cT conversions. However, 5′ methylated cytosines are protected against the bisulfite conversion. This example shows that incorporating varying percentages of 5′-methyl-dCTP into cDNA by reverse transcription and performing bisulfite conversion results in gA conversions and the absence of cT conversions for features that are located on the positive strand in subsequent sequencing library preparations (FIG. 11). Since the cDNA is the reverse complement of the original RNA molecule, gA conversions are expected for positive-strand features and CT for negative-strand features. This indicates that the strategy described in this example efficiently produces cDNA molecules with patterns of errors that can, for example, be used for molecule counting, strandedness identification, and RNA molecule sequence reconstruction.

### Example 9

### Materials and Methods

Single K562 cells were sorted into individual wells of a 384-well plate containing 0.3 μL Smart-seq3 lysis buffer (see: Hagemann-Jensen et al, 2020), with dPTP present at 0.5 mM and each dNTP present at 0.1 mM. Reverse transcription was performed according to the Smart-seq3 protocol (see: Hagemann-Jensen et al, 2020), with a 10-fold volume reduction and the MgCl₂ concentration adjusted to 1.5 mM. FastAP was added to a final concentration of 0.1 U/μL in a total volume of 0.5 μL. The reaction was incubated at 37° C. for 20 minutes and FastAP was inactivated at 72° C. for 10 minutes. cDNA was amplified as described in Example 1 above. The resulting cDNA library was tagmented as described in Example 1 above in quadruplicates to maximise fragment complexity. The resulting libraries were circularised using the MGI App-A conversion kit and sequenced on the MGI DNBSEQ-G400 platform using a StandardMPS PE200 kit.

Data was processed using zUMIs (https://github.com/sdparekh/zUMIs). The option find_pattern ATTGCGCAATG (SEQ ID NO: 5) was specified to identify UMI-containing 5′-reads and all reads were mapped using STAR to perform alignment with the human genome (hg38). STAR settings were changed to allow for up to 20% mismatches. Note that mismatches here correspond to all possible mismatches.

### Results

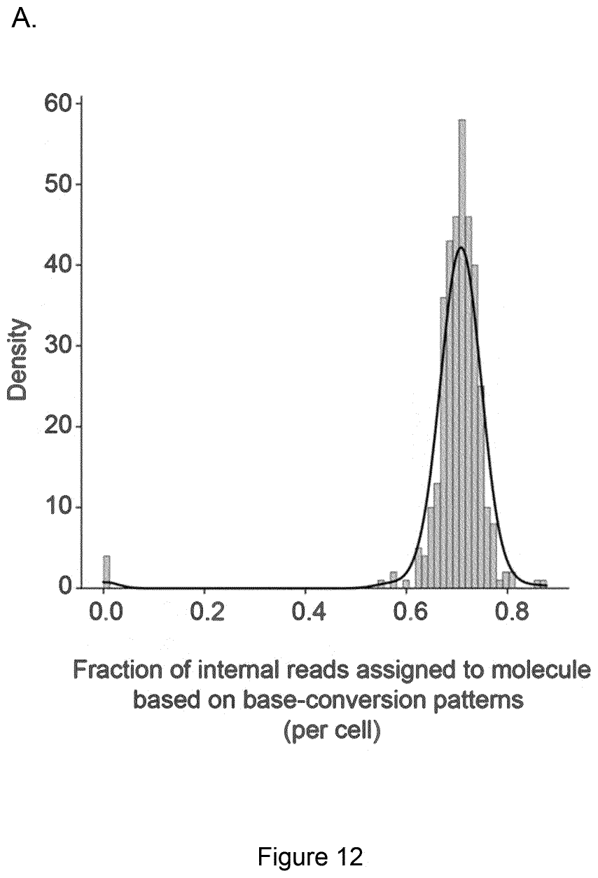

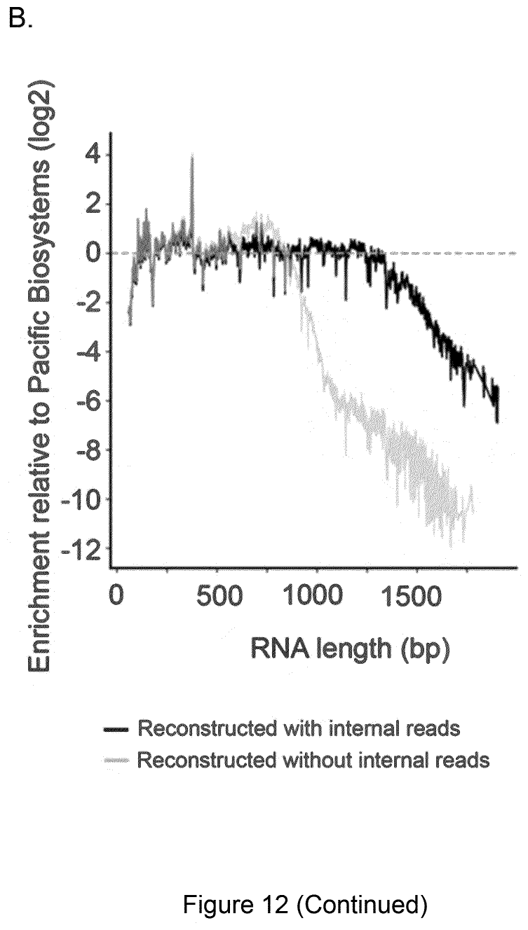

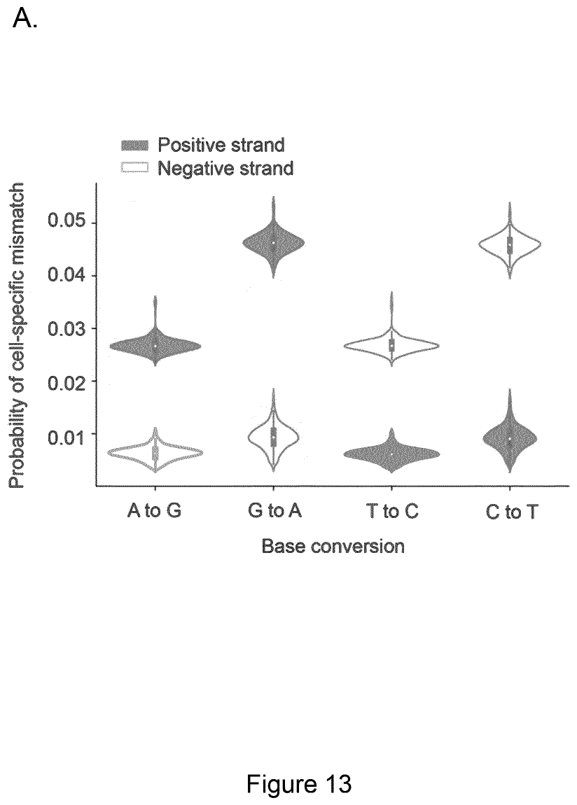

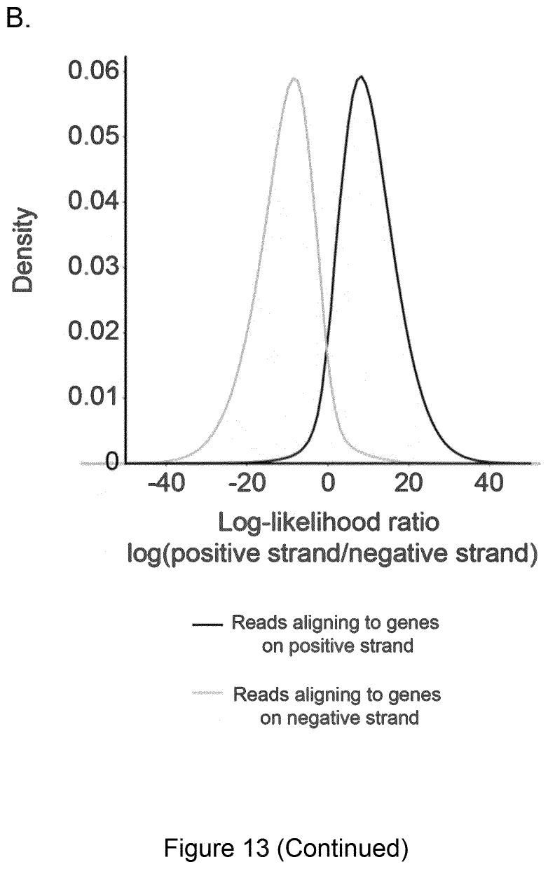

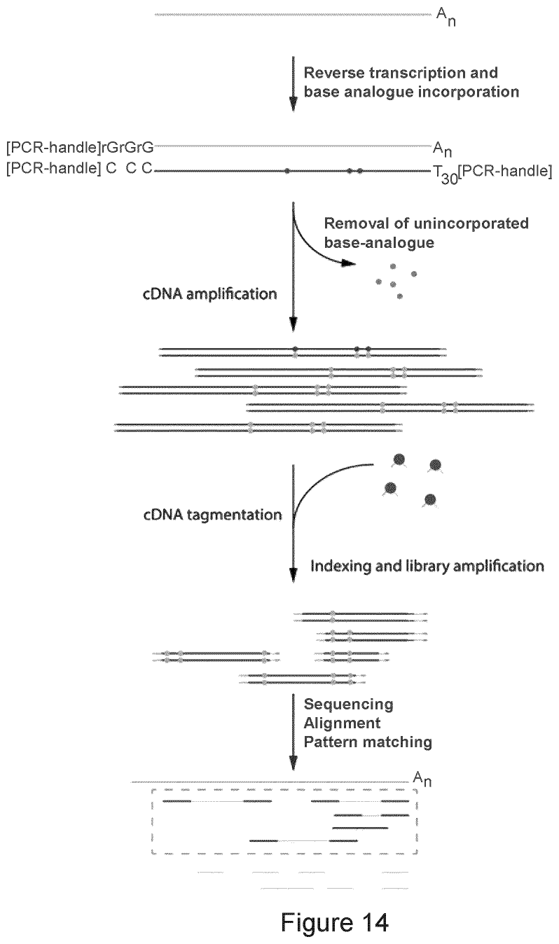

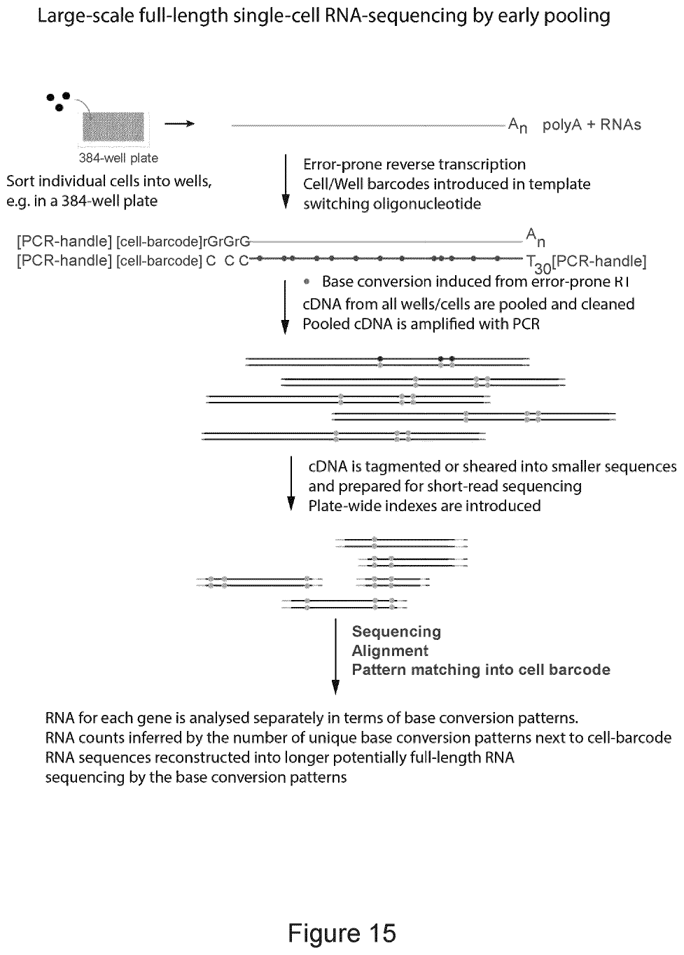

Smart-seq3 data typically consists of ‘UMI reads’ and ‘internal reads’. The UMI-reads contain a UMI and can be linked to individual RNA molecules, with those reads typically corresponding to the 5′ end of the molecule. The patterns introduced during reverse transcription by the methods of the present invention can be used to efficiently assign ‘internal reads’ to the molecule of origin (FIG. 12A). The lengths of the reconstructed molecules are comparable to lengths obtained from long-read sequencing of full-length CDNA (FIG. 12B). As has already been shown in earlier Examples, the base-conversion pattern is unique to the strand-of-origin of the RNA molecule (FIG. 13A). Therefore, in addition to reconstruction, the induced base-conversion patterns can readily be used to identify the strand from which the corresponding RNA was transcribed (FIG. 13B).

### Example 10

### Materials and Methods

Single K562 cells were sorted into a 96-well plate with 0.2 μL lysis buffer containing 1 mM dATP, 0.2 mM dCTP, 1 mM dGTP, 1 mM dTTP, 10 mM dPTP, 0.08% Triton-X100 (Sigma), 1.6 U/μL Recombinant RNAse inhibitor (Takara), cell-barcoded and UMI containing oligo-dT primers (for example: TCGTCGGCAGCGTCAGATGTGTATAAGAGACAGAAGTCTGTACTAT GGNNNNNNNNTTTTTTTTTTTTTTTTTTTTTTTT (SEQ ID NO: 1), 2 μM) and 5 μL Vapor-Lock (Qiagen). Cells were lysed at 72° C. for 10 minutes. An 0.2 μL RT reaction mix (10 mM DTT, 2M Betaine, 12 mM MgCl₂, 0.8 U/μL Recombinant RNAse Inhibitor (Takara), 2× Superscript II RT Buffer and 20 U/μL Superscript II enzyme) was added. Reverse transcription was performed at 42° C. for 90 minutes, followed by 10 cycles of 2 minutes 50° C. and 2 minutes 42° C. and finally a single 85° C. hold for 5 minutes before holding at 4° C. The RT reactions were pooled and purified using Zymo Research Clean & Concentrator DNA purification columns using five volumes of DNA Binding buffer and washed twice using DNA wash buffer and eluted in 20 μL. First-strand cDNA was poly-adenylated using Terminal Deoxynucleotidyl Transferase (TDT) in a 25 μL reaction containing 0.75 U/μL TDT enzyme (Sigma, 20 U/μL), 1.5 mM dATP, 0.55× ThermoPol buffer (NEB) and RNAse H (Invitrogen, 2 U/μL) 0.02 U/μL. TDT reactions were incubated at 37° C. for 1 minute and 15 seconds and at 65° C. for 10 minutes before holding at 4° C. 30 μL 2nd-strand synthesis mix (27.5 μL Terra PCR 2× Direct Buffer, 1.76 μL primer (TCGTCGGCAGCGTCAGATGTGTATAAGAGACAGTTTTTTTTTTTTTTTTT TTTTTTT (SEQ ID NO: 2), 1 μM) and 0.55 μL Terra PCR Direct Polymerase mix (1.25 U/μL, Takara) and 0.19 μL Nuclease-free water) was added to the TDT reaction. The resulting reaction was held at 98° C. for 2 minutes, then at 40° C. for 1 minute, and then ramped at 0.2° C. per second to 68° C., where it was held for 6 minutes. Clean-up was performed with Zymo Research Clean & Concentrator DNA purification columns as above and DNA was eluted in a volume of 20 μL. cDNA amplification was performed in 50 μL reactions (1× Terra PCR Direct Buffer, 0.8 UM Amplification primer (TCGTCGGCAGCGTCAGATGTGTATAAGAGACAG (SEQ ID NO: 3), 0.025 U/μL Terra Direct Polymerase mix). The PCR was performed by denaturing at 98° C. for 2 minutes, then cycling 18 times over 10 seconds denaturation at 98° C., 15 seconds annealing at 65° C. and 6 minutes extension at 68° C. After the 18 cycles, it was held at 68° C. for 5 minutes before holding at 4° C. Amplified cDNA was purified using SPRI beads and tagmented as in Example 8. The resulting library was circularised using the MGI App-A conversion kit as per the manufacturer's instructions and sequenced on an MGI DNBSEQ-G400RS platform using a StandardMPS PE200 kit.

Reads were separated into 3′ cell-barcoded reads (>16 As in read 1 base 1-24), 5′ anchored reads (>16 As in read 1 base 25-48) and internal reads (Neither). Each group was separately processed with zUMIs v2.9.7 (https://github.com/sdparekh/zUMIs) and mapped to STAR hg38 with settings ‘-outFilterMismatchNmax 80--outFilterMismatchNoverLmax 0.4--outSAMattributes MD NH HI AS nM--clip3pAdapterSeq AAAAAAAAAAAAA’ (SEQ ID NO: 4) to allow for a high number of mismatches. The resulting bam files were then merged into one bam file. The reads were then used for molecule reconstruction. For each gene, each read was sorted according to start and end position for positively and negatively stranded genes respectively. First, cell-barcoded reads were grouped according to adjusted mutual information, considering the overlap of eligible bases (G in reference) and overlapping conversions (G>A). If the base calling quality of the read at a given position was below a Phred score of 15, that position was not considered for the adjusted mutual information calculation. Reads were added to an existing group if the adjusted mutual information exceeded 0.2 for a unique group. If there were no groups above 0.15, the read forms a new group. If there were multiple matches above 0.2, the read was discarded. The conversion pattern for a molecule group was determined by requiring at least 20% of reads with a Phred score above 14 to have the conversion in that position.

When all cell-barcoded reads had been grouped according to conversion pattern, the non-barcoded reads were used. Each non-barcoded read was compared to molecule patterns across all cells in the sample. If the read had more than 0.3 in adjusted mutual information to one unique molecule group, that read, and its corresponding conversion pattern, was added to that molecule group. If there are no matches or more than one match (>0.3 adj. mutual information), that read is discarded. This process was repeated twice.

Once non-barcoded reads have been assigned to a molecule, all the reads were written to a new bam file with its new molecule group as a tag. If the read was not barcoded, the inferred cell of origin is also added. The reads were then merged into one reconstructed molecule read using stitcher.py (https://github.com/AntonJMLarsson/stitcher.py).

### Results

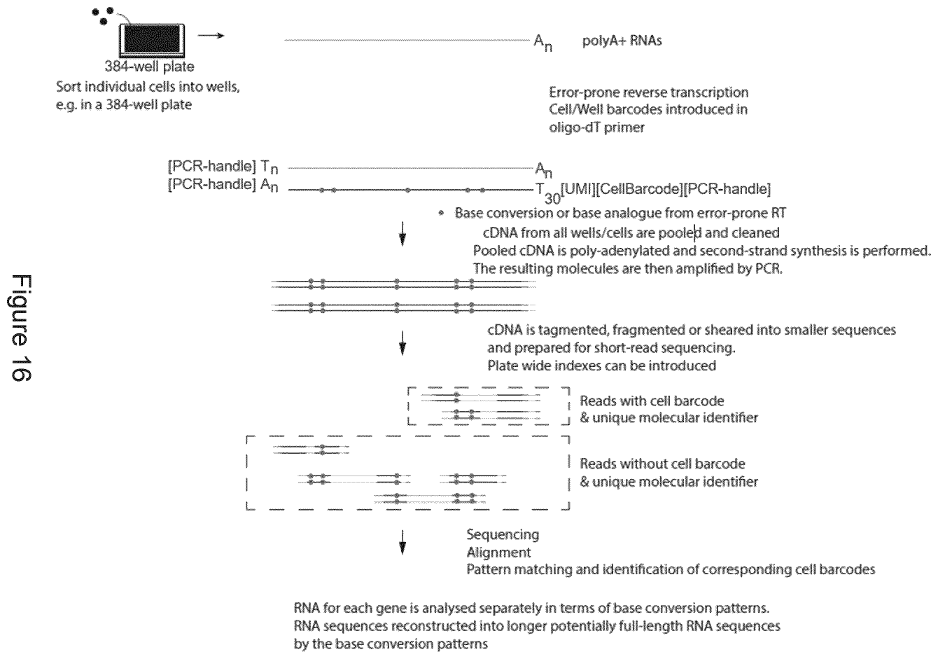

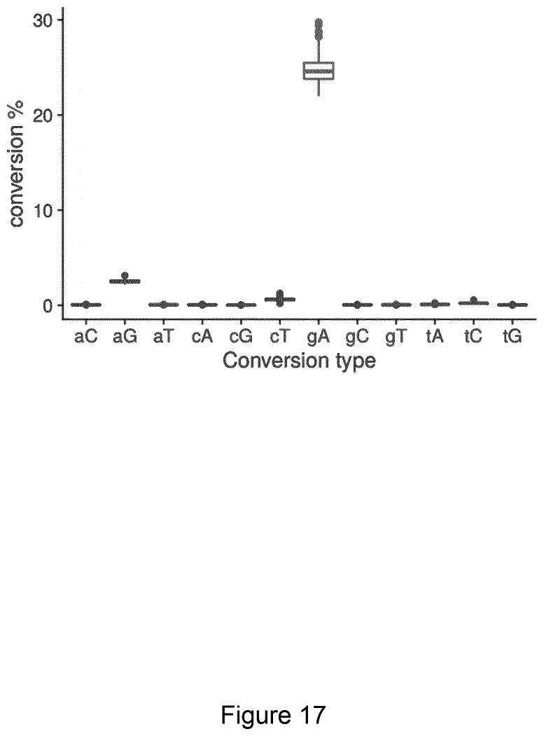

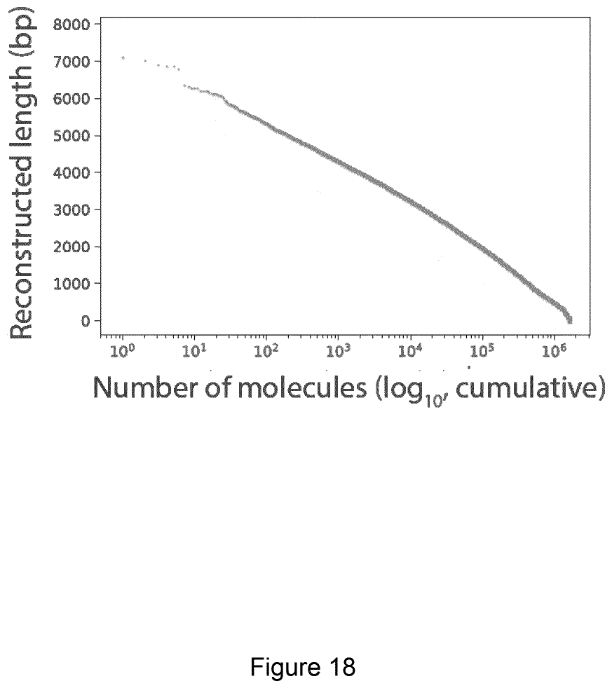

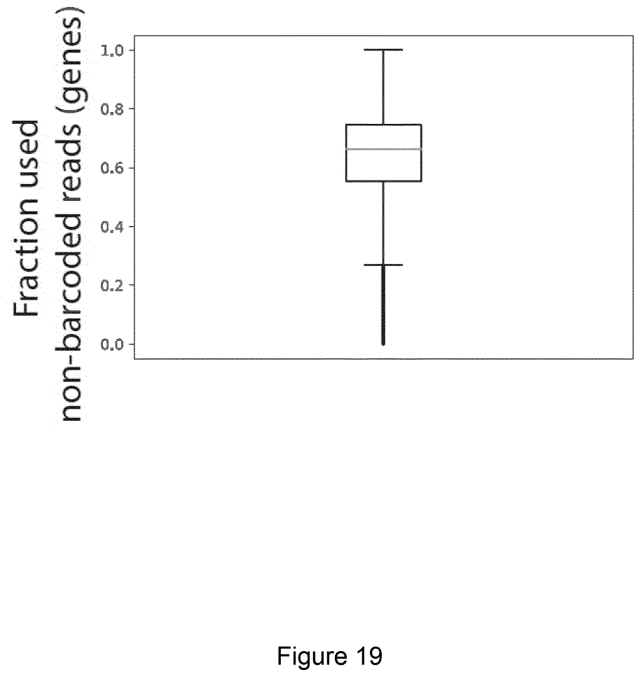

The addition of dPTP into a library preparation strategy that relies on 5′ A-tailing instead of template switching gives rise to 3′ reads that have cell barcodes and UMIs as well as reads that do not have barcodes or UMIs, but that all originate from an RNA molecule which was reverse transcribed with a primer that introduced both a cell barcode and UMI (see FIG. 16). This type of approach results in very high conversion rates (i.e. >20% for the desired G>A conversion) (see FIG. 17), which allows efficient RNA molecule reconstruction. As shown in FIGS. 18 and 19, even reads without cell barcodes can be effectively linked to the cell barcode that was added during reverse transcription of the original RNA molecule through the molecule-specific base-conversion patterns.

### Example 11

### Materials and Methods

Primary mouse fibroblasts were cultured in the presence of 4-thio-uridine (Sigma, 200 μM) for 2 hours and single cells were sorted into 0.3 μL lysis buffer (2.5 U/μL Recombinant RNAse Inhibitor (Takara), 0.2% Triton-X100) in 3 μL Vapor-Lock (Qiagen). 0.3 μL Aklylation reaction mix was added (final reaction concentrations: 50 mM Tris-HCL pH 8, 45% DMSO, 10 mM iodoacetamide) and reactions were incubated at 50° C. for 10 minutes. 0.4 μL Quenching mix was added (final concentrations: 35 mM DTT, 2 mM dNTPs, 2.4 μM Smart-seq3 oligo-dT (Hagemann-Jensen et al, 2020) and 1.6 U/μL Recombinant RNAse inhibitor (Takara)). Samples were then incubated at 72° C. for 10 minutes. 3 μL Reverse Transcription mix was added (33.3 mM Tris-HCL pH 8, 46.7 mM NaCl, 1.3 mM GTP, 3.3 mM MgCl₂, 6.7% PEG (MW 8000), 2.7 mM DTT, 0.5 U/μL Recombinant RNAse Inhibitor (Takara), 2.7 μM Smart-seq3 Template Switching Oligo (Hagemann-Jensen et al, 2020), 2.7 U/μL Maxima H-minus RT enzyme). Reverse Transcription and the remaining library preparation was performed as described in Hagemann-Jensen et al, 2020. Library circularisation and sequencing was performed as in Example 10.

Reads were processed with zUMIs (https://github.com/sdparekh/zUMIs). The option find_pattern ATTGCGCAATG (SEQ ID NO: 5) was specified to identify UMI-containing 5′-reads and mapped to mm10 with STAR settings ‘-outFilterMismatchNmax 40--outFilterMismatchNoverLmax 0.25--outSAMattributes MD NH HI AS nM XS--outSAMstrandField intronMotif--clip3pAdapterSeq CTGTCTCTTATACACATCT’ (SEQ ID NO: 6).

The reads were then used for molecule reconstruction. For each gene, each read was sorted according to start and end position for positively and negatively stranded genes, respectively. First, cell-barcoded reads were grouped according to adjusted mutual information, considering the overlap of eligible bases (T in reference) and overlapping conversions (T>C). If the base calling quality of the read at a given position was below a Phred score of 15, that position was not considered for the adjusted mutual information calculation. Reads were added to an existing group if the adjusted mutual information exceeded 0.2 for a unique group. If there were no groups above 0.15, the read was used to form a new group. If there were multiple matches above 0.2, the read was discarded. The conversion pattern for a molecule group was determined by requiring at least 20% of reads with a Phred score above 14 to have the conversion in that position. All the reads were written to a new bam file with its new molecule group as a tag. If the read was not barcoded, the inferred cell of origin is also added. The reads were then merged into one reconstructed molecule read using stitcher.py (https://github.com/AntonJMLarsson/stitcher.py).

### Results

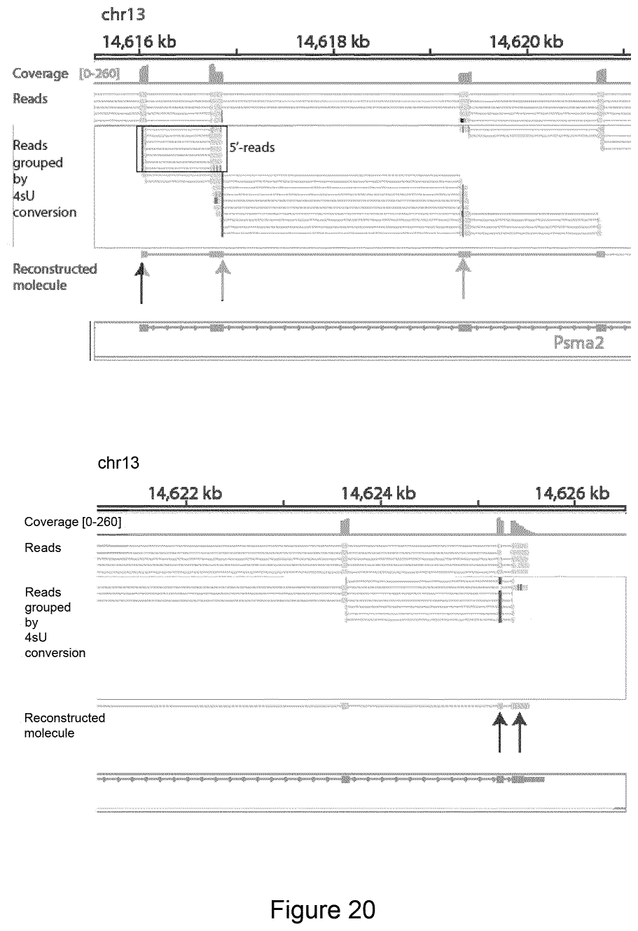

Newly produced RNA molecules in single mouse fibroblasts were labelled with 4-thiouridine U and read out as base conversion corresponding to RNA molecules using and updated version of NASC-seq (see Materials and Methods of Hendriks et al. 2019. Nat. Commun., 10 (1): 3138). The results of this Example demonstrate that the base conversion patterns that are introduced using this method can be used to effectively reconstruct the RNA molecule sequence (FIG. 20). This approach shows that by labelling newly produced RNA in cells with 4-thio-uridine, subsequently treating with iodoacetamide, and preparing a sequencing library, molecule-identifying patterns were created that could be used to reconstruct the sequences of the original RNA molecules present.

### Example 12

### Materials and Methods

Single HEK293T cells were sorted to a 96-well plate and lysis and reverse transcription was performed as described in Example 10. The pooled and purified first-strand cDNA was then poly-adenylated and cleaned up again using a Zymo Research clean & concentrator column before being split into 4 reactions. Second strand synthesis was then performed using the Terra PCR Direct Polymerase Buffer and PCR Direct Polymerase Mix with 0.03 μM primer (TCGTCGGCAGCGTCAGATGTGTATAAG AGACAGTTTTTTTTTTTTTTTTT) (SEQ ID NO: 2). The concentration of dATP in two of the reactions was then increased by 1 mM by adding extra dATP. The four reactions were then cleaned up using a Zymo Research clean & concentrator column. The remainder of the library preparation process was then performed as in Example 10. Library circularisation was performed as in Example 10 and sequencing was performed on a DNBSEQ-G400RS using StandardMPS PE150 chemistry.

The resulting data were processed as in Example 10 without performing any reconstruction. Error rates were directly calculated from the zUMIs output bam files. Cells for which less than 400,000 bases were covered by sequencing reads were removed from the analysis.

### Results

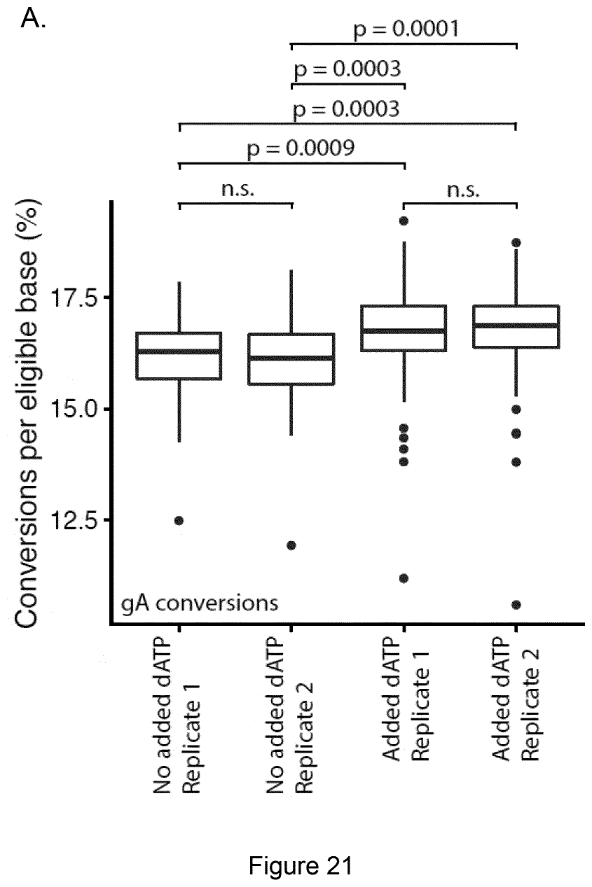

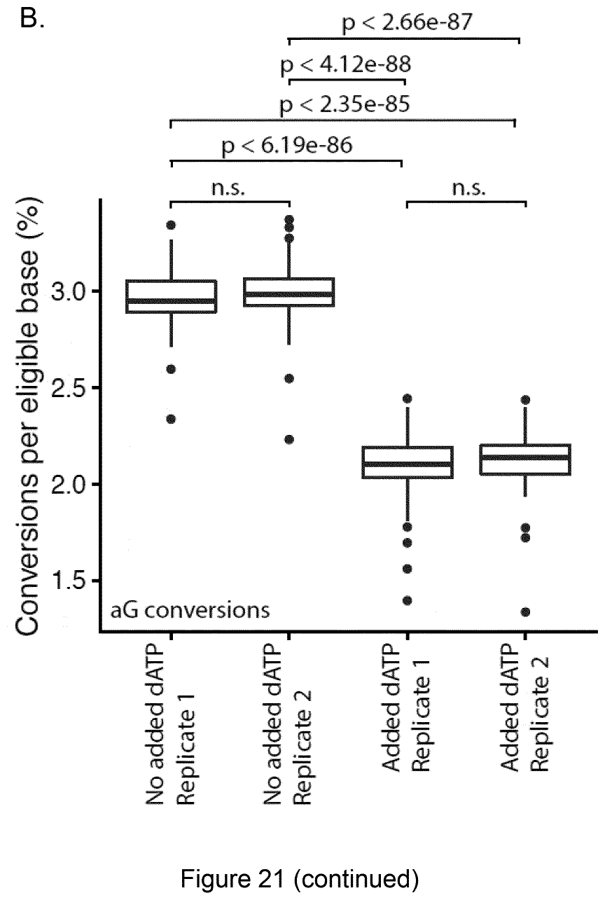

Adding extra dATP during second-strand synthesis, and thereby creating a suboptimal balance of dNTP concentrations, results in the favouring of G-to-A conversions instead of A-to-G conversions as can be seen in FIG. 21. FIG. 21 shows a significant difference in the conversion rates between both replicates of the two conditions groups (two-sided t-tests) in response to the inclusion of additional dATP during second-strand synthesis.

1. A method for determining the number of copies of one or more RNA molecule in a population of RNA molecules, the method comprising:
providing a population of RNA molecules;
subjecting the population of RNA molecules to error-prone reverse transcription, to generate a population of DNA molecules in which each DNA molecule comprises one or more base-conversions relative to the corresponding RNA molecule, and wherein each DNA molecule comprises a molecule-specific base-conversion pattern; and
using the molecule-specific base-conversion pattern to determine the number of copies of the one or more RNA molecule in a population.

2-41. (canceled)

42. The method according to claim 1, wherein the method further comprises:
determining the sequence of overlapping fragments of DNA molecules in the population;
determining the partial or full-length sequence of the DNA molecules in the population by assembling the sequence of overlapping fragments based on the molecule-specific base-conversion pattern in the DNA molecules;
determining, from the partial or full-length sequence of the DNA molecules in the population, the sequence of the RNA molecules which correspond to the DNA molecules; and
determining, from the sequence of the RNA molecules which correspond to the DNA molecules, the number of copies of one or more RNA molecule in the population.

43. A method for determining the sequence of one or more RNA molecule in a population of RNA molecules, the method comprising:
providing a population of RNA molecules;
subjecting the population of RNA molecules to error-prone reverse transcription, to generate a population of DNA molecules in which each DNA molecule comprises one or more base-conversion relative to the corresponding RNA molecule and wherein each DNA molecule comprises a molecule-specific base-conversion pattern; and
using the molecule-specific base-conversion pattern to determine the sequence of the RNA molecule that corresponds to the one or more DNA molecule.

44. The method of claim 43, wherein the method further comprises:
determining the sequence of overlapping fragments of DNA molecules in the population;
determining the sequence of one or more DNA molecule in the population by assembling the sequence of overlapping fragments based on the molecule-specific base-conversion pattern of the DNA molecule; and
determining the sequence of the RNA molecule which corresponds to the one or more DNA molecule.

45. The method according to claim 1, wherein the population of RNA molecules comprises RNA molecules with different sequences and/or RNA molecules with the same sequence.

46. The method according to claim 1, wherein the population of RNA molecules analysed comprises 1 to 100,000,000,000 individual RNA molecules.

47. The method according to claim 1, wherein the population of RNA molecules comprises messenger RNA (mRNA), precursor mRNA (pre-mRNA), antisense RNA (asRNA) or precursors thereof, enhancer RNA or precursors thereof, long non-coding RNA (lncRNA) or precursors thereof, microRNA (miRNA) or precursors thereof, ribosomal RNA (rRNA) or precursors thereof, transfer RNA (tRNA) or precursors thereof, histone RNA or precursors thereof, small nucleolar RNA (snoRNA) or precursors thereof, small nuclear RNAs (snRNA) or precursors thereof, mitochondrial RNA or precursors thereof, viral RNA, transposon RNA, synthetic RNA, in vitro transcribed RNA, or any combination thereof.

48. The method according to claim 1, wherein subjecting the population of RNA molecules to error-prone reverse transcription comprises introducing one or more base-conversion into each DNA molecule at a total rate of about 0.5% to about 99.5%.

49. The method according to claim 1, wherein subjecting the population of RNA molecules to error-prone reverse transcription comprises reverse transcription in the presence of one or more base analogue.

50. The method according to claim 49, wherein the one or more base analogue is selected from the group consisting of: 2′-deoxy-P-nucleoside-5′-triphosphate (dPTP); 8-Oxo-2′-deoxyguanosine-5′-triphosphate (8-oxo-GTP); 2-Thiothymidine-5′-Triphosphate (2-thioTTP), 5-Formyl-2′-deoxyuridine-5′-triphosphate, 5-Propynyl-2′-deoxycytidine-5′-triphosphate, 5-Iodo-2′-deoxycytidine-5′-triphosphate, 5-Propargylamino-2′-deoxyuridine-5′-triphosphate, or combinations thereof.

51. The method according to claim 1, wherein subjecting the population of RNA molecules to error-prone reverse transcription comprises reverse transcription in the presence of a sub-optimal amount of one or more dNTP base.

52. The method according to claim 1, wherein the method comprises incorporating one or more base analogue into the one or more RNA molecule in the population of RNA molecules prior to providing the population of RNA molecules.

53. The method according to claim 52, wherein the one or more base analogue is 4-thio-uridine.

54. The method according to claim 1, further comprising chemically modifying the population of RNA molecules prior to subjecting the population of RNA molecules to reverse transcription.

55. The method according to claim 54, wherein chemically modifying the population of RNA molecules comprises alkylating the population of RNA molecules.

56. The method according to claim 1, wherein subjecting the population of RNA molecules to error-prone reverse transcription further comprises chemically modifying the population of DNA molecules generated by reverse transcription.

57. The method according to claim 54, wherein the chemical modification comprises a deamination reaction.

58. The method according to claim 1, wherein subjecting the population of RNA molecules to error-prone reverse transcription comprises reverse transcription using an error-prone reverse transcriptase enzyme.

59. The method according to claim 42, wherein determining the sequence of overlapping fragments of DNA molecules in the population comprises amplifying the population of DNA molecules to generate one or more amplicon of each DNA molecule in the population.

60. The method according to claim 59, wherein the step of amplifying the population of DNA molecules comprises high-fidelity amplification.

61. The method according to claim 59, wherein the step of amplifying the population of DNA molecules comprises PCR amplification.

62. The method according to claim 59, wherein the step of amplifying the population of DNA molecules is performed in the absence of a base analogue.

63. The method according to claim 59, wherein at least the first cycle of the step of amplifying the population of DNA molecules is performed in the presence of a sub-optimal amount of one or more dNTP base.

64. The method according to claim 42, wherein determining the sequence of overlapping fragments of DNA molecules in the population comprises fragmenting the population of DNA molecules to generate overlapping fragments.

65. The method according to claim 64, wherein the step of fragmenting the population of DNA molecules comprises tagmentation, DNA shearing, and/or enzymatic fragmentation.

66. The method according to claim 64, wherein the fragments are about 50 base pairs to about 1500 base pairs in length.

67. The method according to claim 42, wherein determining the sequence of overlapping fragments of DNA molecules in the population comprises sequencing overlapping fragments of the population of DNA molecules.

68. The method according to claim 67, wherein sequencing comprises a short-read sequencing method.

69. The method according to claim 42, wherein determining the partial or full-length sequence of the DNA molecules in the population comprises:
assigning overlapping fragments to an RNA molecule present in the population of RNA molecules based on their alignment to some or all of the sequence of that RNA molecule;
sorting the assigned fragments based on the position in the RNA molecule at which those fragments align; or
both assigning overlapping fragments to an RNA molecule present in the population of RNA molecules based on their alignment to some or all of the sequence of that RNA molecule and sorting the assigned fragments based on the position in the RNA molecule at which those fragments align.

70. The method according to claim 42, wherein determining the sequence of the RNA molecules which correspond to the DNA molecules comprises:
comparing the partial or full-length sequence of DNA molecules in the population to a reference sequence; and
identifying mismatches corresponding to one or more base-conversion.

71. The method according to claim 42, wherein determining the number of copies of one or more RNA molecule in the population comprises identifying the number of unique molecule-specific base-conversion patterns that correspond to an RNA molecule with a particular sequence in the population of RNA molecules.

72. The method according to claim 1, wherein one or more steps is performed in a droplet-based environment, a plate-based environment, attached to beads, or in-situ.

73. The method according to claim 1, wherein the population of RNA molecules comprises:
one or more sequence variant of the same gene;
one or more allelic variant of the same gene;
one or more splice variant of the same gene;
one or more RNA isoforms resulting from alternative use of promoters;
one or more RNA isoforms resulting from alternative use of splice sites; or
one or more RNA isoforms resulting from alternative use of polyadenylation sites.

74. A method for generating base-conversions in one or more polynucleotide molecule in a population of polynucleotide molecules, the method comprising:
providing a population of polynucleotide molecules, wherein one or more of the polynucleotide molecules comprises one or more base analogue; and
amplifying the population of polynucleotide molecules to generate one or more amplicon of each polynucleotide molecule in the population, wherein the amplifying is performed in the presence of a sub-optimal amount of one or more dNTP base.

75. A kit for performing error-prone reverse transcription, wherein the kit comprises:
a reverse transcriptase enzyme;
one or more base analogue; and,
instructions for use.

76. The kit according to claim 75, wherein the one or more base analogue is selected from the group consisting of: 2′-deoxy-P-nucleoside-5′-triphosphate (dPTP); 8-Oxo-2′-deoxyguanosine-5′-triphosphate (8-oxo-GTP); 2-Thiothymidine-5′-triphosphate (2-thioTTP), 5-Formyl-2′-deoxyuridine-5′-triphosphate, 5-Propynyl-2′-deoxycytidine-5′-triphosphate, 5-Iodo-2′-deoxycytidine-5′-triphosphate, 5-Propargylamino-2′-deoxyuridine-5′-triphosphate, or combinations thereof.

77. The kit according to claim 75, wherein the reverse transcriptase is an error-prone reverse transcriptase.

78. The method according to claim 59, wherein determining the sequence of overlapping fragments of DNA molecules in the population comprises fragmenting the one or more amplicon of each DNA molecule in the population to generate overlapping fragments.

79. The method according to claim 78, wherein the step of fragmenting the one or more amplicon of each DNA molecule in the population comprises tagmentation, DNA shearing, and/or enzymatic fragmentation.
